SEQUENCE LISTING (3 sequences)

SEQ ID NO: 1 - protein, 1202 aa, Homo sapiens
AAAPFPDRPPAHLVSSRRSAPPGSREPRGTGHLHPPLGVSGSSWCLACVSWMPCGFSPSPVAHHLVPGPPDTPAQQLRCGWTVGGWLLSLVRGLLPCLPPGARTAEGPIMVLAGPLAVSLLLPSLTLLVSHLSSSQDVSSEPSSEQQLCALSKHPTVAFEDLQPWVSNFTYPGARDFSQLALDPSGNQLIVGARNYLFRLSLANVSLLQATEWASSEDTRRSCQSKGKTEEECQNYVRVLIVAGRKVFMCGTNAFSPMCTSRQVGNLSRTTEKINGVARCPYDPRHNSTAVISSQGELYAATVIDFSGRDPAIYRSLGSGPPLRTAQYNSKWLNEPNFVAAYDIGLFAYFFLRENAVEHDCGRTVYSRVARVCKNDVGGRFLLEDTWTTFMKARLNCSRPGEVPFYYNELQSAFHLPEQDLIYGVFTTNVNSIAASAVCAFNLSAISQAFNGPFRYQENPRAAWLPIANPIPNFQCGTLPETGPNENLTERSLQDAQRLFLMSEAVQPVTPEPCVTQDSVRFSHLVVDLVQAKDTLYHVLYIGTESGTILKALSTASRSLHGCYLEELHVLPPGRREPLRSLRILHSARALFVGLRDGVLRVPLERCAAYRSQGACLGARDPYCGWDGKQQRCSTLEDSSNMSLWTQNITACPVRNVTRDGGFGPWSPWQPCEHLDGDNSGSCLCRARSCDSPRPRCGGLDCLGPAIHIANCSRNGAWTPWSSWALCSTSCGIGFQVRQRSCSNPAPRHGGRICVGKSREERFCNENTPCPVPIFWASWGSWSKCSSNCGGGMQSRRRACENGNSCLGCGVEFKTCNPEGCPEVRRNTPWTPWLPVNVTQGGARQEQRFRFTCRAPLADPHGLQFGRRRTETRTCPADGSGSCDTDALVEDLLRSGSTSPHTVSGGWAAWGPWSSCSRDCELGFRVRKRTCTNPEPRNGGLPCVGDAAEYQDCNPQACPVRGAWSCWTSWSPCSASCGGGHYQRTRSCTSPAPSPGEDICLGLHTEEALCATQACPEGWSPWSEWSKCTDDGAQSRSRHCEELLPGSSACAGNSSQSRPCPYSEIPVILPASSMEEATGCAGFNLIHLVATGISCFLGSGLLTLAVYLSCQHCQRQSQESTLVHPATPNHLHYKGGGTPKNEKYTPMEFKTLNKNNLIPDDRANFYPLQQTNVYTTTYYPSPLNKHSFRPEASPGQRCFPNS

SEQ ID NO: 2 - DNA, 4559 bp, Homo sapiens
gcggccgccccattcccagaccggccgccagcccatctggttagctcccgccgctccgcgccgcccgggagtcgggagccgcggggaaccgggcacctgcacccgcctctgggagtgagtggttccagctggtgcctggcctgtgtctcttggatgccctgtggcttcagtccgtctcctgttgcccaccacctcgtccctgggccgcctgataccccagcccaacagctaaggtgtggatggacagtagggggctggcttctctcactggtcaggggtcttctcccctgtctgcctcccggagctaggactgcagaggggcctatcatggtgcttgcaggccccctggctgtctcgctgttgctgcccagcctcacactgctggtgtcccacctctccagctcccaggatgtctccagtgagcccagcagtgagcagcagctgtgcgcccttagcaagcaccccaccgtggcctttgaagacctgcagccgtgggtctctaacttcacctaccctggagcccgggatttctcccagctggctttggacccctccgggaaccagctcatcgtgggagccaggaactacctcttcagactcagccttgccaatgtctctcttcttcaggccacagagtgggcctccagtgaggacacgcgccgctcctgccaaagcaaagggaagactgaggaggagtgtcagaactacgtgcgagtcctgatcgtcgccggccggaaggtgttcatgtgtggaaccaatgccttttcccccatgtgcaccagcagacaggtggggaacctcagccggactactgagaagatcaatggtgtggcccgctgcccctatgacccacgccacaactccacagctgtcatctcctcccagggggagctctatgcagccacggtcatcgacttctcaggtcgggaccctgccatctaccgcagcctgggcagtgggccaccgcttcgcactgcccaatataactccaagtggcttaatgagccaaacttcgtggcagcctatgatattgggctgtttgcatacttcttcctgcgggagaacgcagtggagcacgactgtggacgcaccgtgtactctcgcgtggcccgcgtgtgcaagaatgacgtggggggccgattcctgctggaggacacatggaccacattcatgaaggcccggctcaactgctcccgcccgggcgaggtccccttctactataacgagctgcagagtgccttccacttgccagagcaggacctcatctatggagttttcacaaccaacgtaaacagcatcgcggcttctgctgtctgcgccttcaacctcagtgctatctcccaggctttcaatggcccatttcgctaccaggagaaccccagggctgcctggctccccatagccaaccccatccccaatttccagtgtggcaccctgcctgagaccggtcccaacgagaacctgacggagcgcagcctgcaggacgcgcagcgcctcttcctgatgagcgaggccgtgcagccggtgacacccgagccctgtgtcacccaggacagcgtgcgcttctcacacctcgtggtggacctggtgcaggctaaagacacgctctaccatgtactctacattggcaccgagtcgggcaccatcctgaaggcgctgtccacggcgagccgcagcctccacggctgctacctggaggagctgcacgtgctgccccccgggcgccgcgagcccctgcgcagcctgcgcatcctgcacagcgcccgcgcgctcttcgtggggctgagagacggcgtcctgcgggtcccactggagaggtgcgccgcctaccgcagccagggggcatgcctgggggcccgggacccgtactgtggctgggacgggaagcagcaacgttgcagcacactcgaggacagctccaacatgagcctctggacccagaacatcaccgcctgtcctgtgcggaatgtgacacgggatgggggcttcggcccatggtcaccatggcaaccatgtgagcacttggatggggacaactcaggctcttgcctgtgtcgagctcgatcctgtgattcccctcgaccccgctgtgggggccttgactgcctggggccagccatccacatcgccaactgctccaggaatggggcgtggaccccgtggtcatcgtgggcgctgtgcagcacgtcctgtggcatcggcttccaggtccgccagcgaagttgcagcaaccctgctccccgccacgggggccgcatctgcgtgggcaagagccgggaggaacggttctgtaatgagaacacgccttgcccggtgcccatcttctgggcttcctggggctcctggagcaagtgcagcagcaactgtggagggggcatgcagtcgcggcgtcgggcctgcgagaacggcaactcctgcctgggctgcggcgtggagttcaagacgtgcaaccccgagggctgccccgaagtgcggcgcaacaccccctggacgccgtggctgcccgtgaacgtgacgcagggcggggcacggcaggagcagcggttccgcttcacctgccgcgcgccccttgcagacccgcacggcctgcagttcggcaggagaaggaccgagacgaggacctgtcccgcggacggctccggctcctgcgacaccgacgccctggtggaggacctcctgcgcagcgggagcacctccccgcacacggtgagcgggggctgggccgcctggggcccgtggtcgtcctgctcccgggactgcgagctgggcttccgcgtccgcaagagaacgtgcactaacccggagccccgcaacgggggcctgccctgcgtgggcgatgctgccgagtaccaggactgcaacccccaggcttgcccagttcggggtgcttggtcctgctggacctcatggtctccatgctcagcttcctgtggtgggggtcactatcaacgcacccgttcctgcaccagccccgcaccctccccaggtgaggacatctgtctcgggctgcacacggaggaggcactatgtgccacacaggcctgcccagaaggctggtcgccctggtctgagtggagtaagtgcactgacgacggagcccagagccgaagccggcactgtgaggagctcctcccagggtccagcgcctgtgctggaaacagcagccagagccgcccctgcccctacagcgagattcccgtcatcctgccagcctccagcatggaggaggccaccggctgtgcagggttcaatctcatccacttggtggccacgggcatctcctgcttcttgggctctgggctcctgaccctagcagtgtacctgtcttgccagcactgccagcgtcagtcccaggagtccacactggtccatcctgccacccccaaccatttgcactacaagggcggaggcaccccgaagaatgaaaagtacacacccatggaattcaagaccctgaacaagaataacttgatccctgatgacagagccaacttctacccattgcagcagaccaatgtgtacacgactacttactacccaagccccctgaacaaacacagcttccggcccgaggcctcacctggacaacggtgcttccccaacagctgataccgccgtcctggggacttgggcttcttgccttcataaggcacagagcagatggagatgggacagtggagccagtttggttttctccctctgcactaggccaagaacttgctgccttgcctgtggggggtcccatccggcttcagagagctctggctggcattgaccatgggggaaagggctggtttcaggctgacatatggccgcaggtccagttcagcccaggtctctcatggttatcttccaacccactgtcacgctgacactatgctgccatgcctgggctgtggacctactgggcatttgaggaactggagaatggagatggcaagagggcaggcttttaagtttgggttggagacaacttcctgtggcccccacaagctgagtctggccttctccagctggccccaaaaaaggcctttgctacatcctgattatctctgaaagtaatcaatcaagtggctccagtagctctggattttctgccagggctgggccattgtggtgctgccccagtatgacatgggaccaaggccagcgcaggttatccacctctgcctggaagtctatactctacccagggcatccctctggtcagaggcagtgagtactgggaactggaggctgacctgtgcttagaagtcctttaatctgggctggtacaggcctcagccttgccctcaatgcacgaaaggtggcccaggagagaggatcaatgccacaggaggcagaagtctggcctctgtgcctctatggagactatcttccagttgctgctcaacagagttgttggctgagacctgcttgggagtctctgctggcccttcatctgttcaggaacacacacacacacacactcacacacgcacacacaatcacaatttgctacagcaacaaaaaagacattgggctgtggcattattaattaaagatgatatccagtctcc

SEQ ID NO: 3 - protein, 13 aa, Artificial Sequence
  Description of Artificial Sequence Synthetic Peptide
SVRIGLPKEESRN

(19) United States
(12) Patent Application Publication
(10) Pub. No.: US 2005/0042636 A1
(43) Pub. Date: Feb. 24, 2005

(54) Compositions and methods for detecting abnormal cell proliferation

(76) Inventors: Lance A. Liotta, North Potomac, MD (US); Elizabeth Woodhouse, Bethesda, MD (US); Emanuel Petricoin, Dunkirk, MD (US)

Correspondence Address: EDWARDS & ANGELL, LLP, P.O. BOX 55874, BOSTON, MA, 02205, US

(21) Appl. No.: 10/835,531

(22) Filed: Apr. 28, 2004

Related U.S. Application Data
(60) Provisional application No. 60/466,154, filed on Apr. 28, 2003.

(51) Int. Cl. C12Q 1/68

(52) U.S. Cl. 435/6

(57) ABSTRACT

The invention provides compositions for the detection and treatment of cells misexpressing (e.g., overexpressing) or expressing altered forms of SEMA 5 as well as methods for using the compositions. In a further aspect, high throughput screens are provided for identifying modulators of SEMA 5

### FIELD OF THE INVENTION

The invention relates to compositions useful in therapeutic, diagnostic and screening for abnormal cell proliferation associated with misexpression or alteration of Sema 5 gene products and methods for using such compositions.

### BACKGROUND

Cancer is the second leading cause of death in the United States, after heart disease (Boring, et al., CA Cancer J Clin., 43:7 (1993)). Cancer is characterized primarily by an increase in the number abnormally proliferating cells in a tissue or in the blood stream and the generation of malignant cells which spread via the blood or lymphatic system to regional lymph nodes and to distant sites, in a process known as metastasis.

Cancer metastasis is a complex multi-step process, thereby hindering the identification of molecules functionally required for this lethal process. In the past, the involvement of particular genes in metastasis has been inferred from correlation studies, in which the genome of patients who have cancer or who are at risk for cancer has been screened for alterations that might be linked to the phenotype of abnormal cellular proliferation.

Despite the numerous examples of isolated antigens and other biomarkers shown to be associated with various cancers, the usefulness of such tools for therapeutic diagnostic, prognostic and other detection applications are limited, for example, in that they have been shown to be ineffective, unreliable, lacking in sensitivity and/or predictiveness, and the like. Thus, there exists a continuing need to identify polypeptides, antigenic epitopes and nucleic acid sequences encoding other biomarkers associated with cancer and to develop these as therapeutic targets.

### SUMMARY OF THE INVENTION

In one aspect, the invention provides a method of detecting abnormal cellular proliferation such as cancer in a subject comprising detecting expression of a Sema 5 gene product, wherein overexpression of the Sema 5 gene product compared to expression of Sema 5 in a reference sample from a subject with normally proliferating cells provides an indication of increased risk of abnormal cellular proliferation in the subject. The subject is preferably a mammal such as a human being.

Detecting is generally performed by obtaining a sample from the subject, contacting the sample with a molecular probe that specifically binds to a Sema 5 gene product (e.g., RNA or protein), and identifying the presence and/or amount of binding complexes formed between the molecular probe and Sema 5 gene product. In one aspect, the Sema 5 gene product comprises a nucleic acid. In another aspect, the Sema 5 gene product comprises a polypeptide. The molecular probe can be an antibody or antigen binding fragment thereof or a nucleic acid molecule.

The invention also provides a method of screening for a subject at risk for developing or having cancer comprising an altered or misexpressed Sema 5 gene product or an amplified Sema 5 gene, comprising the steps of: obtaining cells from an individual at risk for or having cancer and detecting the presence of an altered or misexpressed Sema 5 gene product or an amplified Sema 5 gene; and correlating the presence of the altered or misexpressed Sema 5 gene product or amplified Sema 5 gene with risk for developing or having cancer.

The invention further provides a method of screening for polymorphisms in a Sema 5 gene associated with increased risk of having or developing cancer, comprising obtaining a biological sample from a subject with increased risk for cancer or having cancer, and identifying the presence or absence of a polymorphism in a Sema 5 gene in the subject. Preferably, a population of individuals is screened.

In another aspect, the invention provides an isolated immune effector cell that specifically recognizes a SEMA 5 antigen. The immune effector cell is useful for targeting abnormally proliferating cells overexpressing SEMA 5. In a further aspect, the invention provides a vaccine composition comprising at least one SEMA 5 antigen or nucleic acid molecule encoding at least one SEMA 5 antigen and an adjuvant for enhancing an immune response (e.g., such as a cytokine). Preferably, the vaccine composition comprises a vaccine viral vector comprising the nucleic acid molecule. Multivalent vaccines are also encompassed within the scope of the invention comprising a plurality of different SEMA 5 antigens or one or more nucleic acid molecules expressing a plurality of different SEMA 5 antigens. The immune effector cells and vaccine compositions can be used to generate an immune response against abnormally proliferating cells. Accordingly, in one aspect, the invention comprises administering to a subject, an effective amount of these compositions. Efficacy of the method can be monitored by monitoring one or more of: T cell proliferation in response to SEMA 5 antigens, SEMA 5 specific cytolytic responses, production of protective antibodies against SEMA 5 antigens, and the like.

In one aspect, the invention provides a therapeutic antibody composition comprising an antibody or antigen binding fragment thereof which specifically binds to a SEMA 5 antigen, wherein the antibody is stably associated with at least one effector molecule for targeting and/or killing a cell. The effector molecule can be a toxin or a molecule that specifically binds to a cancer cell, or a combination thereof. By administering therapeutically effective amounts of the antibody or antigen binding fragments, an abnormally proliferating cell can be selectively targeted and destroyed.

In another aspect, the invention provides a composition for treating abnormal cellular proliferation in a subject comprising a molecule that decreases or prevents expression of a SEMA 5 gene product. For example, the molecule can be an an antisense molecule, a ribozyme, an iRNA, or an anti-SEMA 5 antibody. The compositions can be used in a method of inhibiting cellular proliferation. In one aspect, the method comprises administering a composition to a cell in a therapeutically effective amount for inhibiting or elimating abnormal cellular proliferation. Administering can be accomplished by a variety of means, e.g., systemically, by topical administration, by injection into a tumor, etc. The composition can be administered alone or as an adjunct to a cancer therapy (e.g., such as chemotherapy).

In still another aspect, the invention provides a library of variant Sema 5 molecules (nucleic acids, peptides or polypeptides) for screening for agonists or antagonists of SEMA 5. The variant molecules may be expressed in the form of fusion proteins for phage display. Agonist or antagonist forms can be assayed for functionally by monitoring their effect on Sema 5 suppression of a neoplastic phenotype induced by mutations in the gene l(2)gl of Drosophila.

The invention also provides a kit comprising a molecular probe that specifically binds to a Sema 5 gene product and a biological sample comprising an abnormally proliferating cell or a portion thereof which overexpresses Sema 5. In another aspect, the kit comprises a biological sample comprising a normally proliferating cell or portion thereof. Biological samples include, but are not limited to: a cell lysate, a cell culture sample, or a section of a tissue or cell.

In a further aspect, the invention provides a method for identifying a mutated gene which is a modulator of a neoplastic phenotype, comprising: introducing a tissue into an adult fly, wherein the tissue comprises a reporter sequence and a mutated modulator gene and wherein the tissue is derived from a mutant fly comprising a mutated l(2)gl gene capable of conferring a neoplastic phenotype and a mutated Sema 5 gene which suppresses the neoplastic phenotype of the mutated l(2)gl gene. Cell proliferation is evaluated by monitoring the expression of the reporter sequence in cells from different tissues in the adult fly. One or more of: an increase in the numbers of different tissues expressing the reporter sequence and an increase in the level of reporter sequence expressed in one or more tissues, identifies the mutated modulator gene as a modulator of a neoplastic phenotype. In a different aspect of the invention, the adult fly comprises a mutated modulator gene and the tissue which is introduced into the adult fly comprises a mutated l(2)gl gene capable of conferring a neoplastic phenotype and a mutated sema 5 gene which suppresses the neoplastic phenotype of the mutated l(2)gl gene.

In yet another aspect, the invention provides a method for identifying a mutated gene which is a modulator of a neoplastic phenotype. The method comprises introducing a tissue into an adult fly, wherein the tissue comprises a reporter sequence and overexpresses a Sema 5 gene. Either the tissue or the adult fly comprises a mutated modulator gene. Changes in a neoplastic phenotype induced by the overexpression of Sema 5 is nonitored, are monitored by measuring one or more of: the numbers of different tissues expressing the reporter sequence and a change in the level of a reporter sequence expressed in one or more tissues, wherein a significant increase or decrease in these parameters identifies the mutated modulator gene as a modulator of the neoplastic phenotype. In one aspect, a modulator is identified which suppresses the neoplastic phenotype. In another aspect, a modulator is identified which enhances the neoplastic phenotype.

Mutations in the l(2)gl gene used in these screens are preferably null or neomorphic mutations. Preferably, the tissue is homozygous for the l(2)gl mutation and/or the mutated Sema 5 gene. Mutations in the Sema 5 genes can be functionally null, hypomorphic, or neomorphic or a conditional mutaton that causes abnormal cellular proliferation under selected conditions.

The tissue used to test neoplasia is preferably obtained from one or more Drosophila larvae, such as brain tissue or imaginal disc tissue.

In one preferred aspect, the reporter sequence is comprised within a P-element. The reporter sequence can be any known in the art, such as, for example, a lacZ gene, GFP gene, BFP gene, or luciferase gene.

### BRIEF DESCRIPTION OF THE FIGURES

The objects and features of the invention can be better understood with reference to the following detailed description and accompanying drawings.

FIGS. 1 A-F illustrate a functional screen for metastasis genes according to one aspect of the invention. FIG. 1A is a schematic diagram showing the use of P-element mutagenesis of a Drosophila genome heterozygous for a mutation in l(2)gl to scan the genome for mutations which are modulators of the neoplastic phenotype of l(2)gl. Adults homozygous for a P-element and heterozyogus for l(2)gl deletion are crossed to generate larvae that are homozygous l(2)gl and homozygous for P-element insertion. Brain tissue from these larvae is transplanted into adults. FIGS. 1B-H show metastasis patterns of l(2)gl insertion, and excision lines (described further below).

FIG. 2A is a schematic illustrating cloning of genomic regions flanking P-element insertion sites. Genomic regions from P-element insertion lines are indicated with arrowheads at P-element insertion site. The 97-2 insertion is located 15.6 kb from the Pi3K59F gene and 16.3 kb from the apontic gene. The 115-1 insertion is 445 bp from the start of the translated region of the pointed gene while the 23-2 insertion is 46 by from the start of the translated region of the semaphorin 5c (sema-5c) gene. FIGS. 2B-C show expression analysis of three modulator genes identified using the HTS system according to one aspect of the invention. FIG. 2B shows PCR amplification of the 23-2 insertion with primers specific for 3′ P-element sequence and genomic sequence flanking the 23-2 insertion. Lane 1: parental genomic DNA, tubulin primers; lane 2: parental genomic DNA, 23-2 insertion primers; Lane 3: 23-2 genomic DNA, tubulin primers; lane 4: 23-2 genomic DNA, 23-2 insertion primers. The tubulin PCR product 655 bp. The 23-2 PCR product is 241 bp. FIG. 2C shows RT-PCR analysis of apontic gene expression. Lane 1: Parental line cDNA, tubulin primers; Lane 2: parental line cDNA, apontic primers; Lane 3: 97-2 cDNA, tubulin primers; Lane 4: 97-2 cDNA, apontic primers. The apontic RT-PCR product is 174 bp. The tubulin RT-PCR product 165 bp. FIG. 2D shows RT-PCR analysis of pointed expression. Lane: 1 115-1 cDNA, tubulin primers; 115-1 cDNA. Lane 2: pointed primers; Lane 3: parental line cDNA, tubulin primers; Lane 4: parental line cDNA, pointed primers. The tubulin RT-PCR product is 165 bp. The ets-like RT-PCR product is 129 bp.

FIGS. 3A-C shows restoration of a neoplastic phenotype by reintroduction of the wild-type modulator gene, Sema-5c, into l(2)gl homozygotes. FIG. 3A shows Western blotting of Drosophila brain extracts with anti-semaphorin antibodies. The parental line expresses Sema-5c (lane 1). The 23-2 insertion line lacks Sema-5c expression (lane 2). The 23-2 excision line restores Sema-5c expression (lane 3). FIG. 3B is a schematic diagram showing Class 5 semaphorin domains. FIG. 3C shows protein microarray analysis of selected signaling proteins in l(2)gl-/l(2)gl- and l(2)gl-/l(2)gl-Sema-5c-/Sema-5c-brain tissues. Wild-type values were subtracted from l(2)gl-/l(2)gl- and l(2)gl-/l(2)gl-Sema-5c-/Sema-5c-values.

FIGS. 4A to B show that SEMA5A protein expression correlates with metastatic potential in murine and human tumor cell lines. FIG. 4A shows Western blot analysis of SEMA5A and P-SMAD1 in 3T3 cells transfected with indicated constructs: Ras+ATX (highly metastatic), Ras (metastatic); Mock-transfected 3T3 cells (non-metastatic). SEMA5A expression was compared in human tumor cell lines: MDA435 (highly metastatic); MDA231 (low metastatic potential), A2058 (non-metastatic). FIG. 4B shows immunostaining of MDA 435 cells with semaphorin antibodies, verifying a cell membrane localization of SEMA5A.

FIG. 5 is a bar graph illustrating that the PI3K inhibitor, LY294002, blocks l(2)gl primary tumor growth in Drosophila but an ERK inhibitor, PD98059, does not. Adult hosts injected with l(2)gl/l(2)gl larval tissue were orally administered drugs for 21 days after injection. Hosts were treated with 0 or 0.56 μg/ml of LY294002 (reduction of tumor size to 7% of untreated) and 0 or 0.56 μml PD98059 (no effect).

FIG. 6 shows the amino acid sequence of human KIAA 1445 (SEMA 5D).

FIG. 7 shows the nucleotide sequence of the coding sequence of human KIAA 1445(Sema 5d).

FIGS. 8(A and B) show the expression of Dpp target gene vestigial is increased in l(2)gl brain tissue compared with wild-type. RT PCR analysis demonstrated elevated vestigial levels (quantitated in proportion to tubulin) (n=3) in l(2) gl tissues compared with wild-type or lgl/lgl; sema-5c/sema-5c. FIG. 8(c) shows a model for the role of TSP-1 repeats in Semaphorin 5c activation of the Dpp pathway.

FIG. 9 shows the expression of human homologs of Semaphorin 5C, including KIAA 1445 (Sema 5D). FIG. 9A shows the expression of SEMA5A and SEMA5D being detected in membrane preparations of A2058 human melanoma cells. FIG. 9B shows the results of an immunohistochemistry assay, which demonstrates membrane localization of SEMA5D in ovarian cancer cells.

### DETAILED DESCRIPTION

The invention provides compositions for the detection and treatment of cells misexpressing (e.g., overexpressing) or expressing altered forms of SEMA 5 as well as methods for using the compositions. In a further aspect, high throughput screens are provided for identifying modulators of SEMA 5.

The practice of the present invention employs, unless otherwise indicated, conventional molecular biology, microbiology, and recombinant DNA techniques within the skill of the art. Such techniques are explained fully in the literature. See, e.g., Maniatis, Fritsch & Sambrook, In Molecular Cloning: A Laboratory Manual (1982); DNA Cloning: A Practical Approach, Volumes I and II, D. N. Glover, ed., (1985); Oligonucleotide Synthesis, M. J. Gait, ed., (1984); Ausubel, et al., (eds.), Current Protocols In Molecular Biology, John Wiley & Sons, New York, N.Y. (1993); Nucleic Acid Hybridization, B. D. Hames & S. J. Higgins, eds., (1985); Transcription and Translation, B. D. Hames & S. I. Higgins, eds., (1984); Animal Cell Culture, R. I. Freshney, ed. (1986); and B. Perbal, A Practical Guide to Molecular Cloning (1984).

Definitions

The following definitions are provided for specific terms which are used in the following written description.

As used herein, “a”, “an,” and ” “the” include plural references unless the context clearly dictates otherwise. For example, a reference to “SEMA 5 polypeptide” includes a plurality of SEMA-5 polypeptides.

As used herein, a “SEMA 5 semaphorin” as distinguished from a “non-SEMA 5 semaphorin” comprises a sema domain, one or more thrombospondin repeats, a transmembrane domain and a cytoplasmic domain and includes class V semaphorins. In another aspect, “a SEMA 5 semaphorin” comprises seven thrombospondin repeats which may be type 1 or type 1-like. A “non-SEMA 5 semaphorin” generally refers to a non-class V semaphorin. A Sema 5d gene product refers to RNA and/or protein products of a Sema 5 gene (i.e., such as Sema 5c, Sema 5b, or Sema 5a genes), including alternatively spliced RNAs, and processed and/or modified SEMA 5 proteins or polypeptides. As used herein, the terms “protein” or “polypeptide” are generally used interchangeably. SEMA 5 polypeptides can include less than the full-length amino acid sequence initially translated by a Sema 5 gene (e.g., polypeptides that arise by cellular processing events or in the generation of recombinant forms of SEMA 5).

An “isolated” or “purified” nucleic acid molecule is one that is separated from other nucleic acid molecules that are present in the natural source of the nucleic acid. Preferably, an “isolated” nucleic acid is free of sequences that naturally flank the nucleic acid (i.e., sequences located at the 5′ and 3′ ends of the nucleic acid) in the genomic DNA of the organism from which the nucleic acid is derived and which are do not contribute to SEMA 5D expression. An “isolated” nucleic acid molecule, such as a cDNA molecule, is substantially free of other cellular material when produced by recombinant techniques or by chemical synthesis methods.

An “isolated” or “purified” protein or polypeptide is one that is separated from other polypeptide molecules that are present in the natural source of the polypeptide and is substantially free of other cellular materials.

As used herein, a “small molecule” is usually less than about 10K in molecular weight and may possess a number of physicochemical and pharmacological properties which enhance cell penetration, allow it to resist degradation and prolong its physiological half-life. Preferably, small molecules are not immunogenic.

“Cells”, “host cells” or “recombinant host cells” are terms used interchangeably herein to refer not only to the particular subject cell, but to the progeny or potential progeny of such a cell.

An “SEMA 5 functional mutation” refers to a mutation within a SEMA 5 gene that results in an altered phenotype (e.g., a change in the expression level and/or activity of a polypeptide encoded by the gene).

“Increased risk” refers to a statistically higher frequency of occurrence of a disease or condition in an individual carrying a particular polymorphic allele of a SEMA 5 gene or overexpressing a SEMA 5 gene in comparison to the frequency of occurrence of the disease or condition in a member of a population that does not carry the particular polymorphic allele or which does not overexpress the SEMA 5 gene.

The term “propensity to disease,” also “predisposition” or “susceptibility” to disease or any similar phrase, means that certain alleles are associated with or predictive of a subject's incidence of developing a particular disease (e.g., such as cancer). Polymorphic alleles associated with a “propensity to disease” are thus over-represented in frequency in individuals with disease as compared to healthy individuals. Thus, these alleles can be used to predict disease even in pre-symptomatic or pre-diseased individuals.

The term “a fly”, unless the context indicates otherwise, generally refers to any stage of a fly's development (e.g., embryo, larva, pupa, adult) and may further refer to a population of flies. When referring to a population of flies, the term “fly” preferably refers to a substantially isogenic population of flies. The term “fly”, “population of flies” and “fly strain” may be used interchangeably in certain contexts.

As used herein, “under transcriptional control” or “operably linked” refers to expression (e.g., transcription or translation) of a polynucleotide sequence which is controlled by an appropriate juxtaposition of an expression control element and a coding sequence. In one aspect, a DNA sequence is “operatively linked” or “operably linked” to an expression control sequence when the expression control sequence controls and regulates the transcription of that DNA sequence. A construct comprising a nucleic acid sequence operably linked to an expression control sequence is referred to herein as an “expression unit” or “expression cassette”.

As used herein, “an expression control sequence” refers to promoter sequences to bind RNA polymerase, enhancer sequences, respectively, and/or translation initiation sequences for ribosome binding. For example, a bacterial expression vector can include a promoter such as the lac promoter and for transcription initiation, the Shine-Dalgarno sequence and the start codon AUG (Sambrook, et al., 1989, supra). Similarly, a eukaryotic expression vector preferably includes a heterologous, homologous, or chimeric promoter for RNA polymerase II, a downstream polyadenylation signal, the start codon AUG, and a termination codon for detachment of a ribosome.

As used herein, a “nucleic acid delivery vector” is a nucleic acid molecule that can transport a polynucleotide of interest into a cell. Preferably, such a vector comprises a coding sequence operably linked to an expression control sequence.

As used herein, “nucleic acid delivery,” or “nucleic acid transfer,” refers to the introduction of an exogenous polynucleotide (e.g., such as an expression cassette) into a host cell, irrespective of the method used for the introduction. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

As used herein, a “a recombinant vaccine vector” refers to a polynucleotide to be delivered into a host cell, either in vivo, ex vivo or in vitro which comprises genomic sequences from a vaccine virus and a heterologous nucleic acid sequence.

As used herein, “an attenuated virus” or a virus having one or more “inactivated virulence associated genes” refers to a virus that is replication deficient or which replicates less efficiently than a wild type virus in a particular host.

As used herein, the term “administering a nucleic acid to a cell” or “administering a vector to a cell” refers to infecting (e.g., in the form of a virus), transducing, transfecting, microinjecting, electroporating, or shooting the cell with the nucleic acid/vector. In some aspects, molecules are introduced into a target cell by contacting the target cell with a delivery cell (e.g., by cell fusion or by lysing the delivery cell when it is in proximity to the target cell).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell, and the progeny may not necessarily be completely identical (in morphology or in genomic or total DNA complement) to the original parent cell due to natural, accidental, or deliberate mutation. A target cell may be in contact with other cells (e.g., as in a tissue) or may be found circulating within the body of an organism.

The term “biologically active fragment”, “biologically active form”, “biologically active equivalent” of and “functional derivative” of a wild-type protein, possesses a biological activity that is at least substantially equal (e.g., not significantly different from) the biological activity of the wild type protein as measured using an assay suitable for detecting the activity.

As used herein, a “patient” is a vertebrate, preferably a mammal, more preferably a human. Mammals include, but are not limited to, murines, non-human primates, humans, farm animals, sport animals, pets, and feral or wild animals.

As used herein, a “subject” refers to any multicellular organism capable of expressing a SEMA 5 polypeptide.

As used herein, a “vaccine” refers to a material that contains or encodes a SEMA 5 antigen and that will provide active immunity to material comprising the antigen, but will not cause disease.

As used herein, a “therapeutically effective amount” refers to an amount sufficient to prevent, correct and/or normalize an abnormal physiological response. In one aspect, a “therapeutically effective amount” is an amount sufficient to reduce abnormal cell proliferation by at least about 10%, at least about 30 percent, more preferably by at least 50 percent, and most preferably by at least 90 percent, or by at least about 1.5 fold, at least about 2-fold, at least about 5 fold, at least about 10-fold, or at least about 20-fold. However, any statistically significant decrease in abnormal cellular proliferation may provide a “therapeutically effective amount”. Abnormal cell proliferation may be measured by monitoring one or more clinically significant features of pathology, such as, for example, tumor size; abnormal biopsy results; presence and extent of abnormally proliferating cells as determined by x-ray, MRI or other medical imaging procedures; hematopoeitic cell counts, histochemical techniques, and by other means well known in the art.

As used herein, a “therapeutically effective amount of a SEMA 5vaccine composition” enhances a beneficial immune response to a SEMA 5 antigen by at least about 30%, as measured by proliferation of T cells in a specific response to a SEMA 5 antigen and/ or by T-cell mediated cytolysis of cells expressing SEMA 5.

The term “proliferation” as used herein means growth and division of cells.

As used herein, the term “normal cells” refers to cells that have a limitation on growth, i.e., a finite number of division cycles.

The term “abnormal cellular proliferation” refers to one or more of a: a removal on a limitation on growth, an inability to remain within appropriate cell boundaries, de-differentiation, and an increase in size in a group of cells at a target site (e.g., a tumor site) which has no normal physiological function.

As used herein, a cell with a “neoplastic phenotype” refers to a phenotype of abnormal, uncontrolled cellular proliferation. Neoplastic cells have a greater ability to cause tumors when injected into a host multicellular organism. A neoplastic phenotype can be recognized by changes in growth characteristics, particularly in requirements for growth factors, and often also by changes in morphology. Neoplastic cells usually proliferate without requiring adhesion to a substratum and usually lack cell to cell inhibition. Neoplastic cells tend to show partial or complete lack of structural organization and functional coordination with the normal tissue, and may be benign or malignant. A neoplastic phenotype may be determined by the induction of at least one tumor in a host organism upon the introduction of cells having a “neoplastic phenotype”.

As used herein, “inhibiting cellular proliferation” refers to slowing and/or preventing the growth and division of cells.

The term “inhibiting metastasis” refers to slowing and/or preventing metastasis or the spread of neoplastic cells to a site remote from a primary growth area.

The term “invasion” as used herein refers to the spread of cancerous cells to surrounding tissues.

As used herein “a growth inhibitory amount” of a modulator compound is an amount capable of inhibiting the growth of a cell, especially a cell with a neoplastic phenotype. In one aspect, a growth inhibitory compound is one which significantly reduces the percentage of the target cells in anyone or all of the cell cycle phases, including G₀, G1, S phase, G2 and mitosis.

As used herein, a “modulator mutation” refers to a mutation in a “modulator gene” which, when disrupted, alters the neoplastic phenotype of a tumorigenic gene. In one aspect, a modulator causes a significant change in one or more of the numbers of tumors induced in a single organism or in a population of organisms, the size of tumors (e.g., numbers of cells which are proliferating abnormally), and/or which changes the amount of metastasis observed, as determined using routine statistical tests, setting p<0.05, or about <0.01. In one aspect, a modulator changes the size of a tumor by at least about 10%. In another aspect, a modulator changes the size of a tumor by at least about 2-fold. In a further aspect, a modulator changes the number of cells proliferating abnormally by at least about 10% or at least about 2-fold. In still another aspect, a modulator alters the amount of metastasis (e.g., as determined by the number of neoplastic cells observed in areas distal to an injection site, or by the numbers of neoplastic cells in different tissue types) by at least about 10% or at least about 2-fold.

A “suppressor of a neoplastic phenotype” or a “suppressor of a tumorigenic gene” causes a significant decrease in one or more of: the numbers of tumors induced in a single organism or in a population of organisms, the size of tumors, and/or which decreases the amount of metastasis observed, as determined using routine statistical tests, setting p<0.05, or about <0.01. An “enhancer of a neoplastic phenotype” or a “enhancer of a neoplastic phenotype” causes a significant increase in one or more of the numbers of tumors induced in a single organism or in a population of organisms, the size of tumors (e.g., numbers of cells which are proliferating abnormally), and/or which increases the amount of metastasis observed, as determined using routine statistical tests, setting p<0.05, or about <0.01.

As used herein, a “differentially expressed” or “misexpressed” gene product, as used herein, refers to a gene transcript or protein that is found in significantly different numbers of copies, or in activated versus inactivated states, in different cell or tissue types of an organism having a tumor or cancer, compared to the numbers of copies or state of the gene product found in the cells of the same tissue in a healthy organism, or in the normal cells of the same tissue in the same organism, as determined using routine statistical methods known in the art (e.g., setting p<0.05, or <0.01).

SEMA 5 Biomarkers of Abnormal Cellular Proliferation

It is a discovery of the instant invention that SEMA 5 molecules (e.g., nucleic acids and polypeptides or peptides encoded by Sema 5 genes) are biomarkers of abnormal cellular proliferation. As disclosed in Example 1, below, the functional role of SEMA 5 genes in abnormal cellular proliferation was identified by screening for homozygous suppressors of the neoplastic phenotype caused by deletions of the lethal (2) giant larvae (l(2)gl) gene in Drosophila malanogaster. The protein encoded by the l(2)gl gene is a myosin binding protein which is expressed in multiple tissues in embryos, in larval salivary glands, imaginal discs, ovary and brain, and in the heads of adult flies. Homologous sequences have been identified in Caenorhabditis elegans, mice, and humans. Amorphic mutations or loss of function mutations are recessive late lethal mutations that die predominantly as larvae, displaying a tumorigenic phenotype. When isolated l(2)gl neoplastic cells from imaginal discs and brain tissue are transplanted into adult flies, they metastasize rapidly upon transplantation into wild-type adult flies.

Disruptions of the Sema-5c gene in Drosophila melanogaster by P-element mutagenesis were found to block both tumorigenic and metastatic phenotypes associated with the l(2)gl deletion. Excision of the P-element resulted in recovery of protein expression and restoration of the neoplastic phenotype.

The Drosophila Sema-5c gene was first identified in a cDNA screen for secreted and transmembrane proteins expressed during embryogenesis (Kopczynski, et al., Proc. Natl. Acad. Sci. USA 95(17): 9973-9978, 1998). A P-element excision mutation in Sema-5c is described by Bahri, et al., Dev. Dyn. 221(3): 322-30, 2001, who reports that the mutations are homozygous viable and show no obvious embryonic phenotypes. Seong et al., Biogerontology 2(3): 209-217, identified a mutation at the Sema 5c locus (P{GS}Sema-5cGS3011) in a screen for conditional longevity mutations, and reports that under particular temperature conditions the mutation confers a longer life span on adult flies. By virtue of its homology to other semaphorins, a role for SEMA-5D in axon guidance has been conjectured. However, a role for SEMA-5D in cellular proliferation has not been not previously contemplated.

The functional role of SEMA 5 semaphorins in cellular proliferation in higher organisms, newly identified herein, was verified by a homology search for genes with sequence homology to the Drosophila Sema 5c gene and expression analysis of such genes. These genes include the mammalian Sema 5 genes- i.e., Sema 5c, Sema 5b, and Sema 5a (in order of decreasing homology to Drosophila Sema 5c). All are class V semaphorins, containing thrombospondin repeats (domains implicated in neurite outgrowth), a sema domain and a transmembrane domain. The human Sema 5d gene sequence was previously identified as the KIAA1445 gene, which was found in a cDNA library of expressed brain sequences designed by the Kazusa DNA Research Institute to identify large genes (see, e.g., Kikuno, et al., Nucleic Acids Res. 30: 166-168, (2002); Ohara, et al., DNA Res., 4, 53-59, (1997)). Although homology to semaphorins was noted, no functional role for the KIAA1445 was established. At the protein level, human SEMA 5D has 99.1 % homology to human SEMA 5B, 93.4% homology to mouse SEMA 5B and 59.1 % homology to human SEMA 5A.

The expression of Sema 5 genes is shown herein to be upregulated in a variety of metastatic cell lines (see, Example 1, below), indicating that Sema 5 gene products provide useful biomarkers of cancer in mammals.

Sema 5 Nucleic Acid Probes

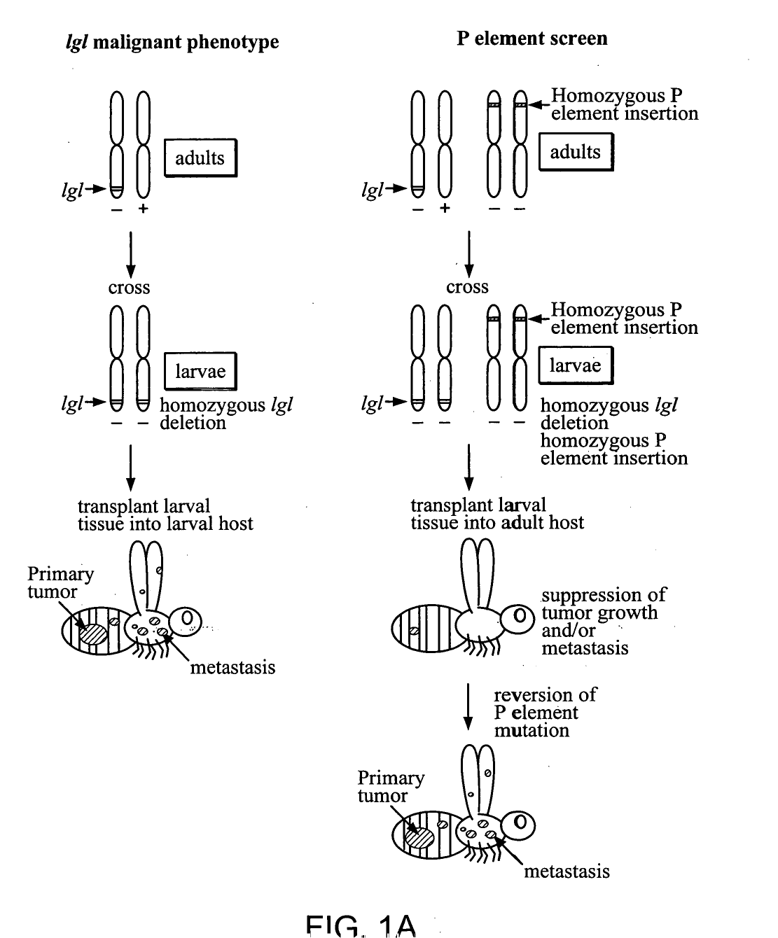

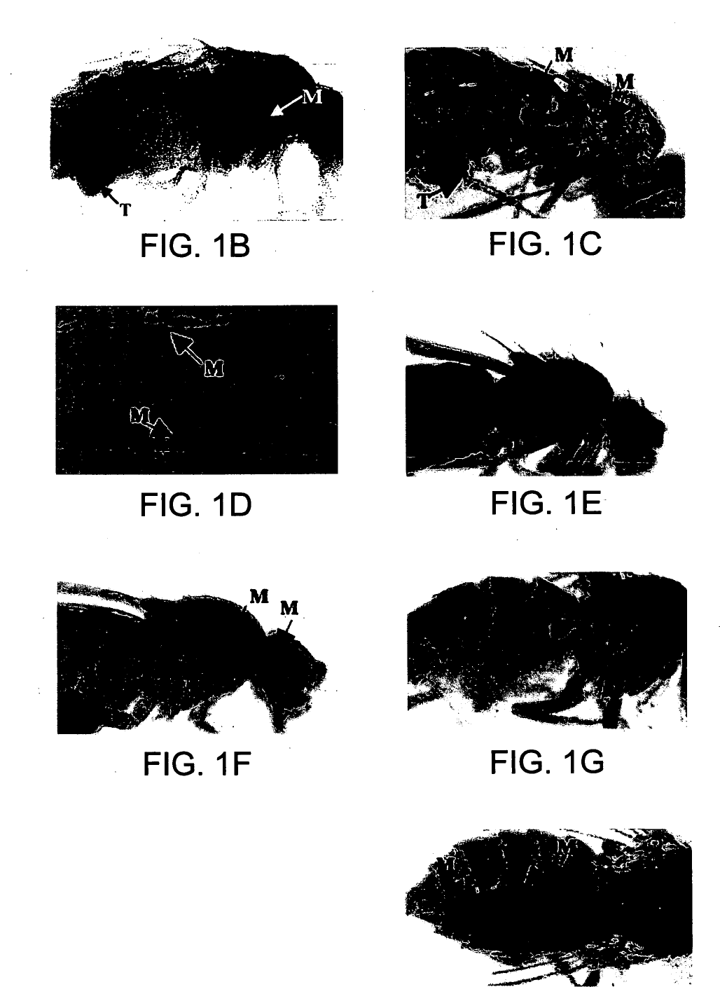

Accordingly, in one aspect, the invention provides molecular probes comprising Sema 5 nucleic acid molecules. Preferably, a probe according to the invention comprises a nucleotide sequence encoding a polypeptide that comprises the amino acid sequence of SEQ ID NO: 1 (as shown in FIG. 1) or a fragment thereof, wherein the fragment specifically hybridizes to a SEMA 5-encoding nucleic acid and not to nucleic acids encoding non-SEMA 5 semaphorins. Nucleic acid probes may be single- or double-stranded and designed to have specificity in PCR, membrane-based hybridization technologies, and the like. As used herein, a “SEMA 5 semaphorin” as distinguished from a “non-SEMA 5 semaphorin” comprises a sema domain, one or more thrombospondin repeats, a transmembrane domain and a cytoplasmic domain and includes class V semaphorins. In another aspect, a SEMA 5 polypeptide encoded by a Sema 5 nucleic acid comprises seven thrombospondin repeats, which may be type 1 or type 1-like.

In a further aspect, a nucleic acid probe according to the invention hybridizes to Sema 5c and Sema 5b, but not to Sema 5a or other non-Sema 5 semaphorins. In a further aspect, the nucleic acid according to the invention hybridizes to Sema d, but not to Sema 5b, Sema 5a or other non-Sema 5 semaphorin nucleic acids.

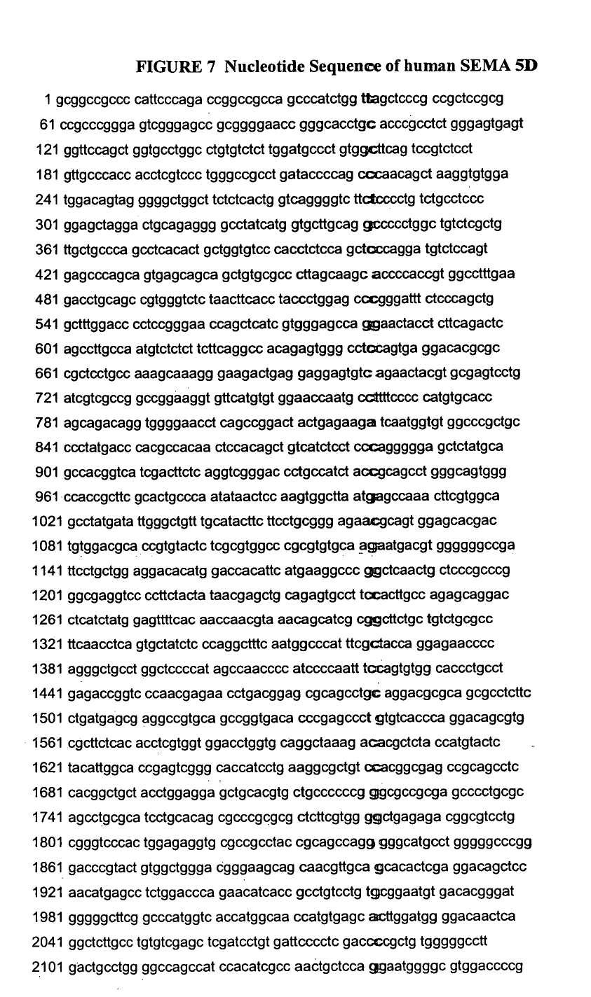

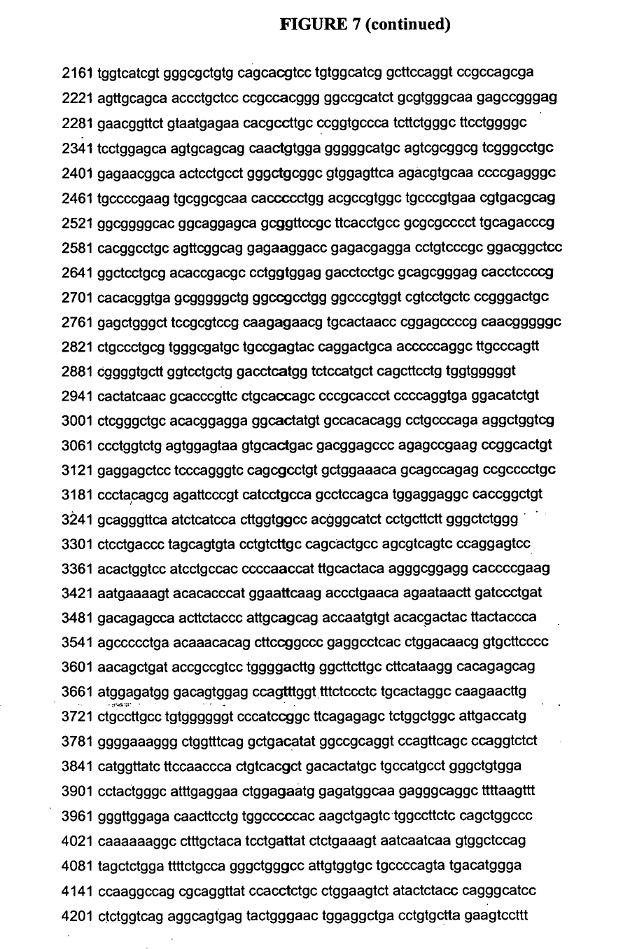

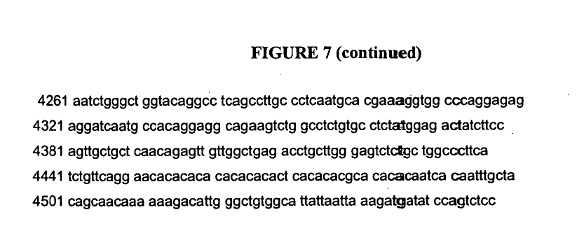

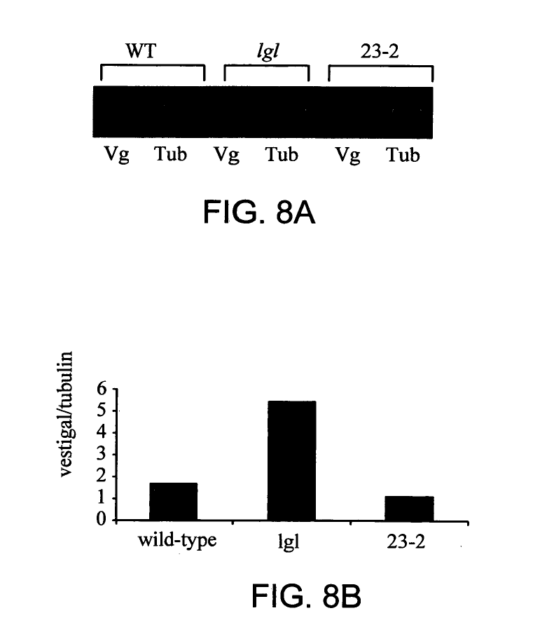

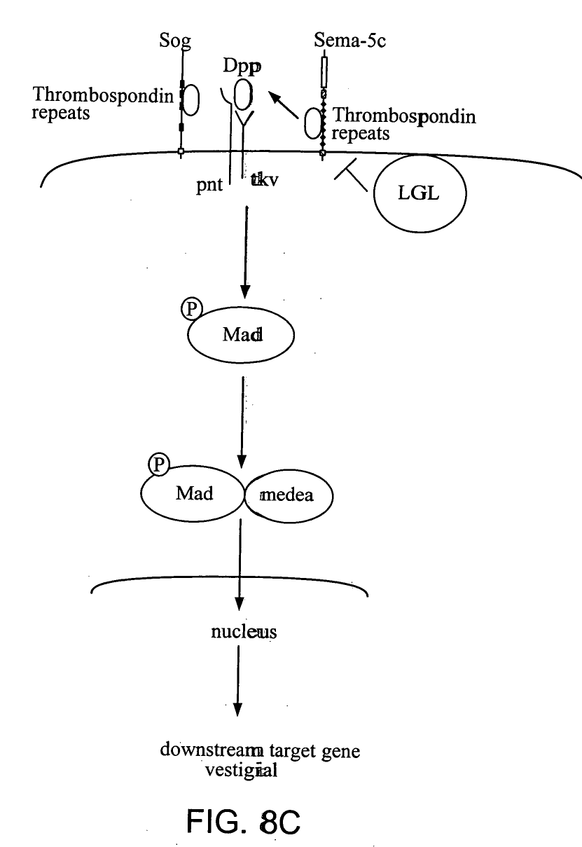

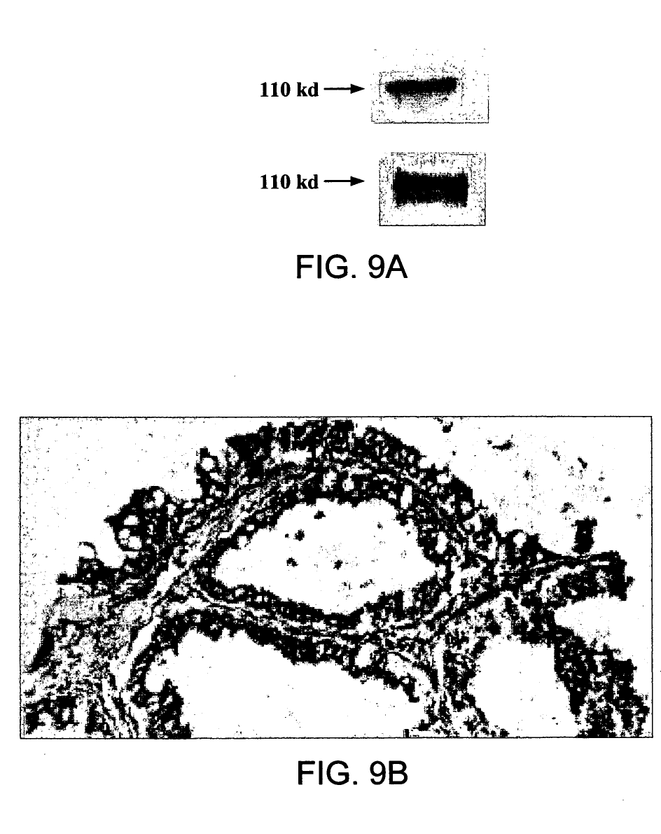

In one preferred aspect, a Sema 5 nucleic acid comprises a sequence encoding a polypeptide according to SEQ ID NO. 1 or antigenic fragment thereof. Preferably, the antigenic fragment is at least about 8 amino acids. In another aspect, a SEMA 5 nucleic acid probe according to the invention comprises a subsequence of the genomic clone gi16163501. In one aspect, the subsequence comprises a coding sequence of the genomic clone. Preferably, the subsequence is capable of hybridizing to the Sema 5d locus on human chromosome 3 under standard in situ hybridization conditions. In a further aspect, a Sema 5 nucleic acid probe according to the invention comprises the sequence of a human, mouse or Drosophila Sema 5, nucleic acid. Such sequences are disclosed in FIG. 7 herein (human Sema 5d) and are additionally disclosed in Genbank under the following, nonlimiting accession numbers: BC002776, AB040878 (human Sema 5d); NM_{—}137449, BI484871, AF198084 (Drosophila Sema 5c); XM_{—}032249 (human Sema 5b); AK078659 (mouse Sema 5b) and NM_{—}003966 (human Sema 5a); AK077021, AK053632, AK043386, AK039101, AK054256, AK046502, AK045236, AK031231, XM_{—}192775, NM_{—}009154 (Mouse Sema 5a); and BG639224, AA264846 (Drosophila Sema 5a).

Variant Sema 5 nucleic acid molecules are also encompassed within the scope of the invention. Preferably, such variants encode SEMA 5 polypeptides that are at least 55% identical to a polypeptide sequence according to SEQ ID NO: 1. In one aspect, SEMA 5 variants encompass natural sequences variants that may exist among individuals within a population due to natural allelic variation and can comprise one or more point mutations, insertions, deleted nucleotides, and the like. Variant Sema 5 sequences include allelic variants of Sema 5a, Sema 5b and Sema 5d. Allelic variants of Sema 5 nucleic acids also include nucleotide sequences that comprise one or more single nucleotide polymorphisms (“SNPs”). A SNP may alter the encoded amino acid sequence shown in FIG. 1 or can reside in a “wobble” section of a codon in a SEMA 5 coding region and thus remain “silent” with no alteration of the encoded SEMA 5 polypeptide sequence. A “nucleic acid encoding a polypeptide comprising an amino acid sequence according to SEQ ID NO. 1” thus encompasses a nucleic acid sequence that comprises the sequence of SEQ ID NO. 2, the coding sequence of gi16163501, as well as nucleic acid molecules that differ because of the degeneracy of the genetic code.

Percent identity and similarity between two sequences (nucleic acid or polypeptide) can be determined using a mathematical algorithm as is known in the art (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987). To determine the percent identity of two nucleic acid sequences or of two amino acid sequences, the sequences are aligned for optimal comparison purposes (e.g., gaps are introduced in one or both of a first and a second nucleic acid or amino acid sequence for optimal alignment and non-homologous sequences can be disregarded for comparison purposes). The percent identity between the two sequences is a function of the number of identical positions shared by the sequences, taking into account the number of gaps, and the length of each gap that needs to be introduced for optimal alignment of the two sequences. The nucleotides or amino acid residues at corresponding nucleotide positions or amino acid positions, respectively, are then compared. When a position in the first sequence is occupied by the nucleotide/amino acid as the corresponding position in the second sequence, then the molecules are identical at that position (as used herein amino acid or nucleic acid “identity” is equivalent to amino acid or nucleic acid “homology”, respectively).

A “comparison window” refers to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

In one aspect, the percent identity between two amino acid sequences (e.g., such as encoded by Sema 5 nucleic acid molecules) is determined using any of: the Needleman and Wunsch algorithm (J. Mol. Biol. 48: 444-453, 1970) which is part of the GAP program in the GCG software package (available at http://www.gcg.com), by the local homology algorithm of Smith & Waterman (Adv. Appl. Math. 2: 482, 1981), by the search for similarity methods of Pearson & Lipman (Proc. Natl. Acad. Sci. USA 85: 2444, 1988) and Altschul, et al. (Nucleic Acids Res. 25(17): 3389-3402, 1997), by computerized implementations of these algorithms (GAP, BESTFIT, FASTA, and BLAST in the Wisconsin Genetics Software Package (available from, Genetics Computer Group, 575 Science Dr., Madison, Wis.), or by manual alignment and visual inspection (see, e.g., Ausubel et al., supra). Gap parameters can be modified to suit a user's needs. For example, when employing the GCG software package, a NWSgapdna.CMP matrix and a gap weight of 40, 50, 60, 70, or 80 and a length weight of 1, 2, 3, 4, 5, or 6 can be used. Examplary gap weights using a Blossom 62 matrix or a PAM250 matrix, are 16, 14, 12, 10, 8, 6, or 4, while exemplary length weights are 1, 2, 3, 4, 5, or 6. The percent identity between two amino acid or nucleotide sequences also can be determined using the algorithm of E. Myers and W. Miller (CABIOS 4: 11-17, 1989) which has been incorporated into the ALIGN program (version 2.0), using a PAM120 weight residue table, a gap length penalty of 12 and a gap penalty of 4.

A nucleic acid probe of the invention can be isolated using standard molecular biology techniques. Using all or a portion of a Sema 5 nucleic acid, variant SEMA 5-encoding sequences and/or homologous Sema 5 sequences and their complements can be isolated using standard hybridization and cloning techniques (e.g., as described in Sambrook et al., (eds.), Molecular Cloning: A Laboratory Manual 2nd Ed., Cold Spring Harbor Laboratory Press, Cold Spring Harbor, N.Y. (1989); and Ausubel, et al., supra).

A nucleic acid probe of the invention can be amplified using cDNA, MRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to Sema 5 nucleotide sequences can be prepared by standard synthetic techniques, e.g., using an automated DNA synthesizer.

Homologs or other related sequences (e.g., paralogs) can be obtained by low, moderate or high stringency hybridization with all or a portion of a human, mouse, or Drosophila sequence as a probe, using methods well known in the art for nucleic acid hybridization and cloning.

An isolated nucleic acid probe of the invention is at least about 6 nucleotides in length, at least about 25 nucleotides in length, at least about 50 nucleotides in length, at least about 100 nucleotides in length, at least about 250, 500 or 2000 nucleotides in length. As used herein, the term “hybridizes under stringent conditions” is intended to describe conditions for hybridization and washing under which nucleotide sequences at least about 55% homologous to each other, at least about 75% homologous, at least about 80% homologous, at least about 90% homologous, or at least about 95% homologous, remain hybridized to each other.

Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for a selected sequence at a defined ionic strength and pH. The Tm is the temperature (under defined ionic strength and pH conditions and nucleic acid concentrations) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nucleotides to 50 nucleotides) and at least about 60° C. for longer probes, primers and oligonucleotides. Stringent conditions may also be achieved with the addition of destabilizing agents, such as formamide.

Stringent conditions are known to those skilled in the art and are described in Ausubel et al., (eds.), Current Protocols In Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non- limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

In another aspect, a nucleic acid sequence that is hybridizable to at least one Sema 5 nucleic acid molecule under moderate stringency conditions is provided. A non-limiting example of moderate stringency hybridization conditions is hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known in the art. See, e.g., Ausubel et al., supra; and John Wiley & Sons, NY, and Kriegler, 1990, Gene Transfer And Expression, A Laboratory Manual, Stockton Press, NY.

In various embodiments, Sema 5 nucleic acids can be modified at the base group, sugar group or phosphate backbone to improve the stability, hybridization, and/or solubility of the molecule. For example, the deoxyribose phosphate backbone of the nucleic acids can be modified to generate peptide nucleic acids (see, Hyrup, et al. Bioorg Med Chem 4: 5-23 (1996)). As used herein, the terms “peptide nucleic acids” or “PNAs” refer to nucleic acid molecules in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Perry-O'Keefe, et al. Proc. Natl. Acad. Sci. USA 93: 14670-675 (1996), for example.

Nucleic acid probes according to the invention can be used to detect transcripts or genomic sequences encoding the SEMA 5 polypeptides. To this end, probes can comprise label groups. Suitable label groups include, but are not limited to: a radioisotope, a fluorescent compound, an enzyme, or an enzyme co-factor. Such probes can be used as a part of a diagnostic or screening test kit for identifying cells or tissue that misexpress Sema 5 gene products and/or which encode altered forms of Sema 5c gene products (e.g., splice variants, mutations and the like). Misexpressed Sema 5 gene products (transcripts and/or proteins) include those that are overexpressed, under expressed, or expressed in ectopic locations. Altered forms include mutant or variant sequences, or inappropriately modified forms of a SEMA 5 polypeptide.

Anti-SEMA 5 Antibodies

The invention additionally provides molecular probes for detecting expression of SEMA 5 polypeptides. In one aspect of the invention, a SEMA 5 polypeptide according to the invention comprises a sema domain, one or more thrombospondin repeats, a transmembrane domain and a cytoplasmic domain. In another aspect, the polypeptide comprises seven thrombospondin repeats, which may be type 1 or type 1-like. In a further aspect, a SEMA 5 polypeptide comprises a sequence according to SEQ ID NO. 1 or a polypeptide that is at least about 55% identical to SEQ ID NO. 1, or a fragment, analog or homolog thereof. Preferably, a SEMA 5 polypeptide fragment is suitable for use as an immunogen to raise anti-SEMA 5 antibodies. In another aspect, a SEMA 5 polypeptide comprises a human, mouse or Drosophila SEMA 5 polypeptide or a fragment, analog, or homolog thereof.

The term “antibody” as used herein encompasses both monoclonal and polyclonal antibodies that fall within any antibody class, e.g., IgG, IgM, IgA, or derivatives thereof. The term “antibody” also includes antibody fragments including, but not limited to, Fab,. F(ab′)₂, and conjugates of such fragments, and single-chain antibodies comprising an antigen recognition site. In addition, the term “antibody” also encompasses humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

Preferably, anti-SEMA 5 antibodies specifically bind to SEMA 5 polypeptides (e.g., such as SEMA 5A, SEMA 5B, and SEMA 5D) and not to non-SEMA 5 semaphorins (i.e., not to non-class V semaphorins). In one preferred aspect, an antibody according to the invention specifically binds to SEMA 5D or SEMA 5B but does not cross-react with a SEMA 5A polypeptide. In another aspect, an antibody according to the invention is capable of distinguishing between SEMA 5A vs. SEMA 5D and SEMA 5B, and/or between SEMA 5D and SEMA 5B. As used herein, the phrase “capable of distinguishing” means that the immunoreactivity of an antibody of the present invention with a target SEMA 5 polypeptide is substantially higher than its immunoreactivity with a non-target SEMA 5 polypeptide so that the binding of the antibody to a target SEMA 5 polypeptide (e.g., SEMA 5D) is readily distinguishable from the binding of the antibody to a non-target SEMA 5 polypeptide based on binding affinities. Preferably, the binding constant of an anti-SEMA 5 antibody from its target differs from the binding constant of the antibody for non-target polypeptides by a magnitude of at least about 2-fold, more preferably at least about 5-fold, even more preferably at least about 10-fold, and most preferably at least about 100-fold.

SEMA 5 proteins can be isolated from cells or tissue sources by an appropriate purification scheme using standard protein purification techniques or can be produced using recombinant DNA techniques as is routine in the art.

For example, a wide variety of host/expression vector combinations may be employed in expressing the nucleic acid sequences of this invention. Useful expression vectors, for example, may consist of segments of chromosomal, non-chromosomal and synthetic nucleic acid sequences.

In one embodiment, prokaryotic cells may be used with an appropriate vector. Prokaryotic host cells are often used for cloning and expression. In a preferred embodiment, prokaryotic host cells include E. coli Pseudomonas, Bacillus and Streptomyces. In a preferred embodiment, bacterial host cells are used to express the nucleic acid molecules of the instant invention. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin and zeocin; auxotrophic markers can also be used.

In other embodiments, eukaryotic host cells, such as yeast, insect, mammalian or plant cells, may be used. Yeast cells, typically S. cerevisiae, are useful for eukaryotic genetic studies, due to the ease of targeting genetic changes by homologous recombination and the ability to easily complement genetic defects using recombinantly expressed proteins. Yeast cells are useful for identifying interacting protein components, e.g. through use of a two- hybrid system. In a preferred embodiment, yeast cells are generally contain an origin of replication suitable for use in yeast and a selectable marker that is functional in yeast. Yeast vectors include Yeast Integrating plasmids (e.g., YIp5) and Yeast Replicating plasmids (the YRp and YEp series plasmids), Yeast Centromere plasmids (the YCp series plasmids), Yeast Artificial Chromosomes (YACs) which are based on yeast linear plasmids, denoted YLp, pGPD-2, 2μ plasmids and derivatives thereof, and improved shuttle vectors such as those described in Gietz et al., Gene, 74: 527-34 (1988) (YIplac, YEplac and YCplac). Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trpl-D1 and lys2-201.

In another embodiment, the host cells may be mammalian cells, which are particularly useful for expression of proteins intended as pharmaceutical agents, and for screening of potential agonists and antagonists of a protein or a physiological pathway. Mammalian vectors intended for autonomous extrachromosomal replication will typically include a viral origin, such as the SV40 origin (for replication in cell lines expressing the large T-antigen, such as COS 1 and COS7 cells), the papillomavirus origin, or the EBV origin for long term episomal replication (for use, e.g., in 293-EBNA cells, which constitutively express the EBV EBNA-1 gene product and adenovirus El A). Vectors intended for integration, and thus replication as part of the mammalian chromosome, can, but need not, include an origin of replication functional in mammalian cells, such as the SV40 origin. Vectors based upon viruses, such as adenovirus, adeno-associated virus, vaccinia virus, and various mammalian retroviruses, will typically replicate according to the viral replicative strategy. Selectable markers for use in mammalian cells include resistance to neomycin (G418), blasticidin, hygromycin and to zeocin, and selection based upon the purine salvage pathway using HAT medium.

Expression in mammalian cells can be achieved using a variety of plasmids, as well as viral vectors (e.g., vaccinia virus, adeno virus, and baculovirus), episomal virus vectors (e.g., bovine papillomavirus), and retroviral vectors (e.g., murine retroviruses), and adenovirus vectors. Useful vectors for insect cells include baculoviral vectors.

Plant cells can also be used for expression, with the vector replicon typically derived from a plant virus (e.g., cauliflower mosaic virus, CaMV; tobacco mosaic virus, TMV) and selectable markers chosen for suitability in plants.

Expression vectors may be either constitutive or inducible. Inducible vectors include either naturally inducible promoters, such as the trc promoter, which is regulated by the lac operon, and the pL promoter, which is regulated by tryptophan, and the MMTV-LTR promoter, which is inducible by dexamethasone. Expression control sequences included in such vectors may include synthetic promoters and/or additional elements that confer inducible control on promoters, such as enhancer sequences. Examples of inducible synthetic promoters are the hybrid Plac/ara-1 promoter and the PLtetO-1 promoter. The PltetO-1 promoter takes advantage of the high expression levels from the PL promoter of phage lambda, but replaces the lambda repressor sites with two copies of operator 2 of the Tn10 tetracycline resistance operon, causing this promoter to be tightly repressed by the Tet repressor protein and induced in response to tetracycline (Tc) and Tc derivatives such as anhydrotetracycline. Vectors may also be inducible because they contain hormone response elements, such as the glucocorticoid response element (GRE) and the estrogen response element (ERE), which can confer hormone inducibility where vectors are used for expression in cells having the respective hormone receptors. To reduce background levels of expression, elements responsive to ecdysone, an insect hormone, can be used instead, with coexpression of the ecdysone receptor.

In one aspect of the invention, expression vectors can be designed to fuse the expressed polypeptide to small protein tags that facilitate purification and/or visualization. Tags that facilitate purification include a polyhistidine tag that facilitates purification of the fusion protein by immobilized metal affinity chromatography, for example using NiNTA resin (Qiagen Inc., Valencia, Calif., USA) or TALONTM resin (cobalt immobilized affinity chromatography medium, Clontech Labs, Palo Alto, Calif., USA). The fusion protein can include a chitin-binding tag and self-excising intein, permitting chitin-based purification with self-removal of the fused tag (IMPACT TM system, New England Biolabs, Inc., Beverley, Mass., USA). Alternatively, the fusion protein can include a calmodulin-binding peptide tag, permitting purification by calmodulin affinity resin (Stratagene, La Jolla, Calif., USA), or a specifically excisable fragment of the biotin carboxylase carrier protein, permitting purification of in vivo biotinylated protein using an avidin resin and subsequent tag removal (Promega, Madison, Wis., USA). As another useful alternative, the proteins of the present invention can be expressed as a fusion protein with glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione. Other tags include, for example, the Xpress epitope, detectable by anti-Xpress antibody (Invitrogen, Carlsbad, Calif., USA), a myc tag, detectable by anti-myc tag antibody, the V5 epitope, detectable by anti-V5 antibody (Invitrogen, Carlsbad, Calif., USA), FLAG® epitope, detectable by anti-FLAGS antibody (Stratagene, La Jolla, Calif., USA), and the HA epitope.

Fusions to the IgG Fc region increase serum half-life of protein pharmaceutical products through interaction with the FcRn receptor (also denominated the FcRp receptor and the Brambell receptor, FcRb), further described in International patent application Nos. WO 97/43316 and WO 97/34631. Plasmid vectors are typically be introduced into chemically competent or electrocompetent bacterial cells.

Purification of recombinantly expressed proteins is well known in the art. See, e.g., Thomer et al. (eds.), Applications of Chimeric Genes and Hybrid Proteins, Part A: Gene Expression and Protein Purification (Methods in Enzymology, Vol. 326), Academic Press (2000); Harbin (ed.), Cloning, Gene Expression and Protein Purification: Experimental Procedures and Process Rationale, Oxford Univ. Press (2001); Marshak et al., Strategies for Protein Purification and Characterization: A Laboratory Course Manual, Cold Spring Harbor Laboratory Press (1996); and Roe (ed.), Protein Purification Applications, Oxford University Press (2001).

Alternatively, antigenic peptides can be synthesized chemically using standard peptide synthesis techniques. This may be desirable when generating antibodies capable of distinguishing between one or more SEMA 5 polypeptides (e.g., SEMA 5A vs. SEMA 5B vs. SEMA 5C). Suitable peptide subsequences of SEMA 5 polypeptides can be readiliy identified by aligning the sequences of SEMA 5A vs. SEMA 5B and SEMA 5C and selecting those regions which comprise unique amino acids.

Regions which are predicted to be highly immunogenic are preferred. Suitable epitope-prediction computer programs include, but are not limited to MacVector from International Biotechnologies, Inc. and Protean from DNAStar. Alternatively, antibodies can be generated by immunizing with an entire SEMA 5 protein and simply selecting for antibodies with the appropriate cross-reactivity as is routine in the art. See, e.g., Harlow and Lane, Antibodies: A Laboratory Manual, Cold Spring Harbor Press, 1988.

SEMA 5 peptide antigens may be coupled to adjuvants to increase the strength of an immune response. Suitable adjuvants or carriers include bovine serum albumin (BSA), ovalbumin, and Tetanus toxoid. Optionally, the antigen is conjugated to a carrier by a coupling agent such as carbodiimide, glutaraldehyde, and MBS. Any conventional adjuvant may be used to boost the immune response of the host animal to the protein complex antigen. Suitable adjuvants known in the art include, but are not limited to Complete Freund's Adjuvant (which contains killed mycobacterial cells and mineral oil), incomplete Freund's Adjuvant (which lacks the cellular components), aluminum salts, MF59 from Biocine, monophospholipid, synthetic trehalose dicorynomycolate (TDM) and cell wall skeleton (CWS) (both available from RIBI ImmunoChem Research Inc., Hamilton, Mont.), non-ionic surfactant vesicles (NISV) (available from Proteus International PLC, Cheshire, U.K.), and saponins. The antigen preparation can be administered to a host animal by subcutaneous, intramuscular, intravenous, intradermal, or intraperitoneal injection, or by injection into a lymphoid organ.

Preferred anti-SEMA 5 antibodies are monoclonal and can be prepared using techniques routine in the art. See, e.g., Kohler and Milstein, Nature 256: 495-497 (1975) and U.S. Pat. No.4,376,110. For example, B-lymphocytes producing a polyclonal antibody against a protein complex of the present invention can be fused with myeloma cells to generate a library of hybridoma clones. The hybridoma population is then diluted and screened for antigen binding specificity and clonal populations of cells. See, e.g., Harlow and Lane, supra. Other techniques include the trioma technique, the human B-cell hybridoma technique (see Kozbor, et al., Immunol Today 4: 72 (1983)) and the EBV hybridoma technique to produce human monoclonal antibodies (see Cole, et al, In: Monoclonal Antibodies And Cancer Therapy, Alan R. Liss, Inc., pp. 77-96 (1985)). Human monoclonal antibodies may be utilized in the practice of the present invention and may be produced by using human hybridomas (see, Cote, et al., Proc Natl Acad Sci USA 80: 2026-2030 (1983)) or by transforming human B-cells with Epstein Barr Virus in vitro (see Cole, et al., In: Monoclonal Antibodies And Cancer Therapy, Alan R. Liss, Inc., pp. 77-96 (1985)).

Recombinant techniques can be used to produce chimeric antibodies (e.g., comprising constant and variable regions are derived from different sources), univalent antibodies, Fab proteins, (see, e.g., U.S. Pat. No. 4,816,567; Munro, Nature 312: 597 (1984); Morrison, Science 229: 1202 (1985); Oi, et al., BioTechniques 4: 214 (1986); and Wood, et al., Nature 314: 446-449 (1985)), and antigen binding fragments (e.g., such as Fv fragments, single-chain Fv fragments (scFv), Fab′ fragments, and F(ab′)2 fragments) (see, e.g., U.S. Patent No. 4,946,778; Skerra and Plückthun, Science 240: 1038-1041(1988); Better, et al., Science 240: 1041-1043 (1988); and Bird, et al., Science, 242: 423-426 (1988)).

According to the invention, techniques can be adapted for the production of single-chain antibodies that specifically bind to SEMA 5 polypeptides (see, e.g., as described in U.S. Pat. No. 4,946,778). In addition, methodologies can be adapted for the construction of F_{ab} expression libraries (see e.g., Huse, et al., Science 246: 1275-1281 (1989)) to allow rapid and effective identification of monoclonal F_{ab} fragments with the desired specificity for a SEMA 5 polypeptide.

In one aspect, SEMA 5 antibodies are partially or fully humanized antibodies. Methods of constructing such antibodies are known in the art. See, e.g., Morrison and Oi, Adv. Immunol. 44: 65-92 (1989). In addition, fully humanized antibodies can be made using transgenic non-human animals. For example, transgenic non-human animals such as transgenic mice can be produced in which endogenous immunoglobulin genes are deleted and which express recombinant antibodies encoded by exogenous immunoglobulin genes, preferably human immunoglobulin genes. See e.g., U.S. Pat. No. 5,530,101; U.S. Pat. No. 5,545,806; U.S. Pat. No. 6,075,181; WO 94/02602; Green, et al., Nat. Genetics 7: 13-21 (1994); and Lonberg et al., Nature 368: 856-859 (1994). The transgenic non-human host animal may be immunized with suitable SEMA 5 antigens to elicit a specific immune response thus producing humanized antibodies.

In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library that is subsequently screened for clones encoding humanized antibodies with desired binding specificities.

Antibodies employed in assays may be labeled or unlabeled. Unlabeled antibodies may be employed in agglutination; labeled antibodies may be employed in a wide variety of assays, employing a wide variety of labels as are known in the art.

Diagnostic Assays

The association between SEMA 5 overexpression and abnormal cell proliferation demonstrates that SEMA 5 molecular probes can be used to screen for the presence, progression of, risk of, and/or reoccurance of abnormal cell proliferation in an animal (e.g., a mammal, such as a human being). The term “overexpression” as used herein means a statistically significant increase in MRNA production or protein production (either expression or accumulation because of increased stability) over that which is normally produced by non-cancerous cells.

In one aspect, a molecular probe capable of specifically binding to a SEMA 5 gene product (RNA or protein) is reacted with a sample and reaction complexes comprising the molecular probe and the SEMA 5 gene product are detected and/or quantified, thereby providing a measure of SEMA 5 gene product in the sample. Suitable samples include, but are not limited to: blood, plasma, urine, lymph, cerebrospinal fluid, serum, semen, breast exudate, saliva, cell or tissue samples, e.g., skin, stroma, vascular epithelium, oral epithelium, vaginal epithelium, cervical epithelium, uterine epithelium, intestinal epithelium, bronchial epithelium, esophageal epithelium, or mesothelium, neural cells, and the like.

The present invention is useful for screening a wide variety of neoplastic diseases, including both solid tumors and hemopoietic cancers. Exemplary neoplastic diseases include carcinomas, such as adenocarcinomas and melanomas; mesodermal tumors, such as neuroblastomas and retinoblastomas; sarcomas, such as osteosarcomas, Ewing's sarcoma, and various leukemias; and lymphomas. Also included within the scope of the invention are tumors of the breast, ovaries, gastrointestinal tract, including the colon and stomach, liver, thyroid glands, prostate gland, brain, pancreas, urinary tract (including bladder), and salivary glands.

Preferably, the molecular probe is detectably labeled, either directly or indirectly. Suitable labels include radionuclides, enzymes, fluorescers, chemiluminescers, enzyme substrates or co- factors, enzyme inhibitors, particles, dyes and the like.

In one aspect, the expression of a plurality of Sema 5 genes is monitored in a single assay. For example, in one aspect, the expression of each of Sema 5a, Sema 5b, and Sema d is monitored.

Cells that are proliferating normally are used to determining base-line expression levels for assays according to the invention. In one aspect, such cells are obtained from normal cells surrounding a site of abnormal proliferation (e.g., a tumor) in a patient. Any statistically significant increase in Sema 5 expression can-have diagnostic value, but generally the MRNA or protein expression will be elevated at least about 2-fold, at least about 3-fold, at least about 5-fold, at least about 10-fold, at least about 50-fold, or at least about 100-fold over expression found in normally proliferating cells.

The particular technique employed for detecting MRNA or protein is not critical to the practice of the invention. For example, gene expression in various tissues may be measured by conventional hybridization techniques. Hybridization formats are well known in the art, and include, but are not limited to, solution phase assays, solid phase assays, mixed phase assays, or in situ hybridization assays. In solution phase hybridizations, both the target nucleic acid and a Sema 5 probe or primer are free to interact in the reaction mixture. In solid phase hybridization assays, either the target or probes are linked to a solid support where they are available for hybridization with complementary nucleic acids in solution. Exemplary solid phase formats include Southern hybridizations and Comparative Genome Hybridization (CGH) assays (e.g., to detect gene amplification), Northern analysis (e.g., to detect overexpression of SEMA 5 mRNAs) dot blots, and the like. In situ techniques are useful for detecting target nucleic acids in chromosomal material (e.g., in metaphase or interphase cells), i.e., to detect amplification of one or more Sema 5 genes. Hybridization assays are described in Singer et al., Biotechniques 4: 230 (1986); Wilkinson, In Situ Hybridization, D. G. Wilkinson ed., IRL Press, Oxford University Press, Oxford; and Nucleic Acid Hybridization: A Practical Approach, Hames, B. D. and Higgins, S. J., eds., IRL Press (1987). For a review of PCR methods and protocols, see, e.g., Innis, et al. eds. PCR Protocols. A Guide to Methods and Application, Academic Press, Inc., San Diego, Calif. (1990). PCR reagents and protocols are also available from commercial vendors, such as Roche Molecular Systems.

Reverse transcription can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described in Romero and Rotbart, in Diagnostic Molecular Biology: Principles and Applications pp.401-406, Persing, et al. eds., Mayo Foundation, Rochester, Minn. (1993); U.S. Pat. No. 5,075,212 and Egger, et al., J. Clin. Microbiol. 33: 1442-1447 (1995), for example.

SEMA 5 polypeptide expression also can be monitored by immunological methods, such as immunohistochemical staining of tissue sections (e.g., such as biopsy tissue sections) and assays of cell cultures or body fluids. Levels of SEMA 5 proteins can be determined in a sample with or without further separation, isolation or purification steps. Immunoassays using antibodies as discussed above include, but are not limited to: competitive binding assays, direct and indirect sandwich assays, and immunoprecipitation assays. See, e.g., U.S. Pat. No.4,376,110 and U.S. Pat. No. 4,486,530, and Zola, Monoclonal Autibodies: A Manual of Techiniques, pp.147-158, CRC Press, Inc., (1987).

Competitive binding assays rely on the ability of a labeled standard to compete with the test sample analyte for binding with a limited amount of antibody. The amount of target protein (encoded by a gene amplified in a tumor cell) in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies may conveniently be separated from the standard and analyte that remain unbound. Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay).

Exemplary immunoassays according to the invention include, but are not limited to: enzyme-linked immunosorbent assays (ELISA), radioimmunoassays (RIA), immunoradiometric assays (IRMA), fluorescent immunoassays, protein A immunoassays, and immunoenzymatic assays (IEMA); flow cytometry and Western blot analysis.

In one aspect, where gene amplification of a SEMA 5 gene is monitored, expression of a SEMA 5 polypeptide is also monitored by one of the assays described above.

Screening for SEMA 5 Polymorphisms Associated with Increased Risk for Abnormal Cellular Proliferation

In another aspect, the invention provides a method for screening for SEMA 5 polymorphisms associated with increase risk for abnormal cellular proliferation, e.g., such as cancer. Suitable assays are well know in the art and include, but are not limited to single stranded conformation analysis (SSCP), restriction fragment length polymorphism (RFLP) analysis, Primed in situ labeling (PRINS), single cell alkaline gel electrophoresis assay (COMET) analysis, heteroduplex analysis, Southern analysis, Northern analysis, denaturing gradient gel electrophoresis (DGGE) analysis; RNase protection assays; the use of proteins which recognize nucleotide mismatches, such as the E. coli mutS protein, and the like. Insertions, deletions and substitutions of SEMA 5 sequences can also be detected by cloning and sequencing. In addition, restriction fragment length polymorphism (RFLP) probes for a Sema 5 gene or surrounding marker genes can be used to identify altered alleles of Sema 5.

High throughput screening for SNPs that affect restriction sites can be achieved by Microtiter Array Diagonal Gel Electrophoresis (MADGE), as described in Day and Humphries, Anal. Biochem. 222: 389-395 (1994), for example. In this assay, restriction enzyme-digested PCR products are loaded onto stackable horizontal gels with wells arrayed in a microtiter format. During electrophoresis, an electric field is applied at an angle relative to the columns and rows of the wells allowing products from a large number of reactions to be resolved.

Alteration of Sema 5 mRNA expression can be detected by any techniques known in the art. These include Northern blot analysis, PCR amplification and RNase protection. Alteration of wild-type genes can also be detected by screening for alteration of wild-type SEMA 5 polypeptide using any of the immunoassays described above. Functional assays can also be performed. In one aspect, such a functional assay comprises transfecting a Sema 5 sequence (in one aspect, a Sema-5c sequence) into Drosophila cells functionally null for the l(2)gl protein and sema 5c genes and monitoring the effect of expression of a variant Sema 5 sequence on the neoplastic phenotype (e.g., by monitoring such parameters as induction of tumors, tumor growth, and metastasis) in assays such as those described further below.

SEMA 5 Therapeutic Nucleic Acids

The overexpression of SEMA 5 nucleic acids and polypeptides in abnormally proliferating cells indicates that SEMA 5 molecules provide useful compositions for regulating cellular proliferation of mammalian cells, such as human cells.

In one aspect, an isolated nucleic acid molecule of the invention comprises a SEMA 5 nucleic acid molecule that is a complement of the nucleotide sequence shown in SEQ ID NO: 2, the coding sequence of gi16163501, or a portion of these sequences. A nucleic acid molecule that is “complementary” is one that is sufficiently complementary to the nucleotide sequence shown in SEQ ID NO 2, the coding sequence of gil6163501, a corresponding RNA, or portions of these sequences so that it can hydrogen bond with little or no mismatches to a Sema 5 nucleotide sequence, forming a stable duplex. In one aspect, a complement is one that contains sufficient sequence complementarity to specifically hybridize to Sema 5 encoding sequences under stringent conditions to detect and distinguish Sema 5 nucleic acids from non-Sema 5 nucleic acids. In another aspect, a complement according to the invention is capable of detecting and distinguishing Sema 5d and Sema 5b from SEMA 5A under stringent hybridization conditions. In another aspect, a complement is capable of distinguishing a Sema 5c nucleic acid molecule from a Sema 5b nucleic acid molecule. In still another aspect of the invention, a complement is capable of priming a polymerization reaction on a nucleic acid template comprising a Sema 5 coding sequence. In a further aspect, the Sema 5 complement is complementary to the coding sequence of a human, mouse, or Drosophila Sema 5 gene.

In a further aspect, a complement is capable of blocking transcription and/or translation of an endogenous cellular Sema 5 gene. Preferably, such complements are complementary to the coding strand of a double-stranded Sema 5 cDNA molecule or complementary to an Sema 5 mRNA sequence. In specific aspects, antisense nucleic acid molecules are provided that comprise a sequence complementary to at least about 10, 25, 50, 100, 250 or 500 nucleotides or an entire SEMA 5 encoding strand. Antisense nucleic acid molecules may comprise modified bases, sugars, or phosphate groups to enhance stability of such molecules in vitro or in vivo as is known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids; e.g., phosphorothioate derivatives and acridine substituted nucleotides can be used.

Examples of modified nucleotides that can be used to generate an antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxylmethylaminomethyl-2-thiouridine, dihydrouracil, inosine, 5-carboxylmethylaminomethyluracil, beta-D- galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentyladenine, uracil-5-oxyacetic acid (v), wybutoxosine, pseudouracil, queosine, 2-thyocytosine, 5-methyl-2-thiouracil, 2-thiouracil, 4-thiouracil, 5- methyluracil, uracil-5-oxyacetic acid methylester, uracil-5-oxyacetic acid (v), 5-methyl-2-thiouracil, 3-(3-amino-3-N-2-carboxypropyl) uracil, and 2,6-diaminopurine. Alternatively, the antisense nucleic acid can be produced biologically using an expression vector into which a nucleic acid has been subdloned in an antisense orientation (i.e., RNA transcribed from the inserted nucleic acid will be of an antisense orientation to a target nucleic acid of interest, described further in the following subsection).

Sema 5 antisense nucleic acid molecules of the invention are typically administered to a subject or generated in situ such that they hybridize with or bind to cellular mRNA and/or genomic DNA encoding a SEMA 5 protein to thereby inhibit expression of the protein, e.g., by inhibiting transcription of a Sema 5 gene and/or translation of Sema 5 mRNA. Routes of administration can vary includes direct injection at a tissue site (e.g., such as injection into a tumor mass). Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using vectors well known in the art. To achieve sufficient intracellular concentrations of antisense molecules, vector constructs in which the antisense nucleic acid molecule is placed under the control of a strong pol II or pol III promoter are preferred.

In yet another embodiment, an antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier, et al., Nucleic Acids Res. 15: 6625-6641 (1987)). The antisense nucleic acid molecule can also comprise a 2′-o-methylribonucleotide (Inoue, et al., Nucleic Acids Res 15: 6131-6148 (1987)) or a chimeric RNA-DNA analogue (Inoue, et al., FEBS Lett. 215: 327-330 (1987)).

Antisense molecules also can comprise PNA molecules. These molecules may be further modified by attaching lipophilic or other helper groups to PNA, by the formation of PNA-DNA chimeras, or by the use of liposomes or other techniques of drug delivery known in the art. PNA-DNA chimeras, comprising PNA seqments and DNA segments, can also be used and generated as described in Finn, et al., Nucl Acids Res 24: 3357-63 (1996), Mag, et al., Nucl Acid Res 17: 5973-88 (1989), and Petersen, et al. Bioorg Med Chem Lett 5: 1119-11124 (1975), for example.

In another embodiment, an enzymatic RNA or ribozyme is designed to target a Sema 5 nucleic acid using methods routine in the art. See, e.g., Kim et al., Proc. Natl. Acad. of Sci. USA 84: 8788 (1987); Haseloff and Gerlach, Nature 334: 585 (1988); and Jefferies et al., Nucleic Acid Res. 17: 1371 (1989). In one aspect, a ribozyme according to the invention comprises a catalytic portion and a Sema 5 complementary sequence which targets the ribozyme to Sema 5 RNA molecules. Methods for selecting a ribozyme target sequence and designing and making ribozymes are generally known in the art. See, e.g., U.S. Pat. No. 4,987,071; U.S. Pat. No 5,496,698; U.S. Pat. No 5,525,468; U.S. Pat. No 5,631,359; U.S. Pat. No 5,646,020; U.S. Pat. No 5,672,511; and U.S. Pat. No 6,140,491. Sema 5 Ribozymes can include hammerhead motifs, hairpin motifs, hepatitis delta virus motifs, group I intron motifs, or RNase P RNA motifs. See, e.g., Rossi, et al., AIDS Res. Human Retroviruses 8: 183 (1992); Hampel and Tritz, Biochemistry 28: 4929 (1989); Hampel, et al., Nucleic Acids Res., 18:299 (1990); Perrotta and Been, Biochemistry 31: 16 (1992); and Guerrier-Takada, et al., Cell 35: 849 (1983). Methods of preparing ribozymes also are described in Usman, et al., J Am. Chem. Soc., 109: 7845-7854 (1987); Scaringe et al., Nucleic Acids Res., 18: 5433-5441 (1990); U.S. Pat. No. 5,652,094; WO 91/03162; WO 92/07065 and WO 93/15187; European Pat. Application No. 92110298.4; Perrault et al., Nature 344: 565 (1990); Pieken, et al., Science 253: 314 (1991); and Usman and Cedergren, Trends in Biochem. Sci. 17: 334 (1992). In one aspect, a Sema 5 mRNA is used to select a catalytic RNA having a specific ribonuclease activity from a pool of RNA molecules. See, e.g., Bartel et al., Science 261: 1411-1418 (1993).

Ribozymes of the present invention may be administered to cells by any known method, e.g., disclosed in International Publication No. WO 94/02595. For example, they can be administered directly to a patient through any suitable route, e.g., by intravenous injection, by topical administration, by direct injection into a tumor (i.e., through an open surgical field or through a medical access device). Alternatively, they may be delivered in encapsulation in liposomes, by iontophoresis, or by incorporation into other vehicles such as hydrogels, cyclodextrins, biodegradable nanocapsules, and bioadhesive microspheres. In addition, Sema 5 nucleic acids may also be delivered using a DNA vector from which the ribozyme RNA is transcribed directly.

In a preferred embodiment, siRNAs are used to down-regulate expression of a Sema 5 gene. The term “short-interfering RNAs (siRNA)” refers to small double-stranded RNAs that interfere with gene expression. siRNAs are intermediates in the processof RNA interference, in which double-stranded RNA silences homologous genes. siRNAs, are typically comprised of two single stranded RNAs of about 21 nucleotides long that form a 19 base pair duplex with about 2 nucleotide 3′ overhangs. Processing of the double stranded RNA by an enzymatic complex (e.g., polymerases), results in cleavage of the double stranded RNA to produce siRNAs. The antisense strand of the siRNA is used by an RNA interference (RNAi) silencing complex to drive MRNA cleavage, thereby promoting mRNA degradation (Fire, et al., Nature 391: 806-811 (1998) and McManus, et al., Nat. Rev. Genet. 3(10): 737-47 (2002)). To silence a Sema 5 gene using siRNAs, a base-pairing region is selected to avoid chance complementarity to an unrelated mRNA. Sequence analysis programs, such as BLAST, can be used to identify suitable sequences in a Sema 5 gene. RNAi silencing complexes have been identified in the art.

Several methods are available for the construction of siRNAs. Such siRNAs can be constructed using T7 phage polymerase. T7 polymerase is used to transcribe individual siRNA sense and antisense strands, which are then annealed to produce a siRNA. The T7 polymerase can also be used to transcribe siRNA strands that are linked in cis, forming a hairpin structure. The transcribed RNAs are comprised of 5′ triphosphate termini or 5′ monophosphates (preferred when silencing Sema 5 genes in mammalian cells).

Therapeutic nucleic acids according to the invention (e.g., antisense molecules, ribozymes, and the like) may further comprise molecules such as peptides (e.g., for targeting host cell receptors in vivo), or agents facilitating transport across the cell membrane (see, e.g., Letsinger, et al., Proc. Natl. Acad. Sci. USA 86: 6553-6556 (1989); Lemaitre, et al., Proc. Natl. Acad. Sci. USA 84:648-652 (1987); and WO88/09810) or the blood-brain barrier (see, e.g., WO 89/10134). In addition, oligonucleotides can be modified with hybridization triggered cleavage agents (see, e.g., Krol et al., BioTechniques 6: 958-976 (1988)) or intercalating agents. (See, e.g., Zon, Pharm. Res. 5: 539-549 (1988)). In one aspect, therefore, a SEMA 5 therapeutic nucleic acid is conjugated to another molecule, such as a peptide, a hybridization triggered cross-linking agent, a transport agent, a hybridization-triggered cleavage agent, and the like.

Therapeutic Antibodies

Therapeutic antibodies are also encompassed within the scope of the invention. In one aspect, a therapeutic antibody is an antibody that neutralizes overexpressed SEMA 5 polypeptides in a cell and normalizes the activity of SEMA 5 polypeptide thereby restoring normal cellular proliferation.

In another aspect, the invention provides therapeutic antibodies comprising an antigen-binding domain which specifically binds to a SEMA 5 polypeptide and which is stably associated with an effector molecule.

Suitable effector molecules comprise one or more of the following functionalities: toxic activity (e.g., cytotoxic activity); anti-angiogenic activity, chemotherapeutic activity; and coagulation activity.

Toxins include, but are not limited to: DNA synthesis inhibitors (e.g., such as daunorubicin, doxorubicin, adriamycin), plant-, fungus- or bacteria-derived toxins such as epipodophyllotoxins; bacterial endotoxin or the lipid A moiety of bacterial endotoxin; ribosome inactivating proteins, such as saporin or gelonin; .alpha.-sarcin; aspergillin; restrictocin; ribonucleases, such as placental ribonuclease; diphtheria toxin and pseudomonas exotoxin; gelonin and/or the A chain toxins, such as ricin A chain.

Molecules with chemotherapeutic activity include, but are not limited to: steroids; cytokines; anti-metabolites, such as cytosine arabinoside, fluorouracil, methotrexate or aminopterin; anthracyclines; mitomycin c; vinca alkaloids; antibiotics; demecolcine; etoposide; mithramycin; and alkylating agents, such as chlorambucil or melphalan, cyclophosphamide; ifosfamide; melphalan (1-sarcolysin); chlorambucil; ethylenimenes; hexamethylmelamine; methylmelamines; alkyl sulfonates; busulfan; nitrosoureas; carmustine (bcnu); lomustine (ccnu); semustine; streptozocin; triazines; dacarbazine (dtic); olecarboxamide; agents that induce apoptosis, and the like,

Molecules with angti-angiogenic activity include, but are not limited to: angiopoietins and angiopoietin fusion proteins, angiostatin, endostatin, laminin peptides, fibronectin peptides, tissue metalloproteinase (TIMP 1, 2, 3, 4); plasminogen activator inhibitors (PAI-1, -2); tumor necrosis factor cc; TGF-β 1; interferons (IFN-α, -.β, gamma), thrombospondin (TSP); and the like.

Preferred coagulation factors for such uses are Tissue Factor (TF) and TF derivatives, such as truncated TF (tTF), dimeric, trimeric, polymeric/multimeric TF, and mutant TF deficient in the ability to activate Factor VII. Other suitable coagulation factors include vitamin K-dependent coagulants, such as Factor II/lIa, Factor VII/VINa, Factor IX/IXa and Factor X/Xa; vitamin K-dependent coagulation factors that lack the Gla modification; Russell's viper venom Factor X activator; platelet-activating compounds, such as thromboxane and thromboxane synthase; and inhibitors of fibrinolysis, such as α-2-antiplasmin. Tumor targeting and treatment with coagulation factor conjugates is described in U.S. Pat. Nos. 5,855,866; 5,965,132; 6,036,955 and 5,877,289; U.S. applications Ser. No. 07/846,349 and U.S. Pat. No. 6,093,399, for example.

Still other effector molecules include targeting agents such as binding partners (e.g., antibodies, ligands, receptors) for cancer specific antigens or tissue-type specific antigens on the surface of a cell. It is noted that an anti-SEMA 5 antibody itself serves as a targeting agent; however additional targeting agents may be provided to enhance the selectivity of a particular treatment regimen. Such agents can include peptides containing the tripeptide R-G-D, that bind specifically to the tumor vasculature, for example. Targets of such agents include intratumoral vasculature cell surface receptors, such as endoglin; TGF β-receptor; E-selectin, P-selectin, VCAM-1, ICAM-1, PSMA, a VEGF/VPF receptor, an FGF receptor, a TIE integrin, pleiotropin, endosialin and MHC Class II proteins.

The preparation of antibodies stably associated with effector molecules is generally well known in the art. See, e.g., U.S. Pat. Nos. 4,340,535, 5,855,866; 5,776,427; 5,863,538; 6,004,554; 5,965,132; 6,051,230; and 5,660,827 for example.

As used herein, “stably associated with” refers to antibodies that are conjugated, linked to, or fused to effector molecules or which are otherwise provided in the same formulation in a manner facilitating joint administration of the antibody and effector molecule to the cell (e.g., such as in a liposome formulation). In one preferred aspect, the effector molecule is coupled to the antibody via a cleavable molecule such as a linker such that the conjugate will remain intact under conditions found everywhere in the body except the intended site of action.

A non-cleavable peptide spacer may be provided to operatively attach the anti-SEMA 5 anitbody and the toxin compound. Toxins that may be used in conjunction with non-cleavable peptide spacers are those that may, themselves, be converted by proteolytic cleavage, into a cytotoxic disulfide-bonded form. An example of such a toxin compound is a Pseudonomas exotoxin compound.

Fusion proteins comprising an anti-SEMA 5 antigen binding portion and an effector polypeptide portion also may be employed. The nucleic acid sequences encoding Sema 5 and effector polypeptide are attached in-frame in an expression vector. Recombinantly produced constructs may be purified and formulated for human administration. Alternatively, nucleic acids encoding the therapeutic agent-targeting agent constructs may be delivered via gene therapy.

In another aspect, the anti-SEMA 5 antibody is linked to the effector molecule via another antibody, i.e., in a heteroconjugate antibody complex or a bispecific antibody. Methods of making such antibodies are disclosed in U.S. Pat. No. 4,676,980; WO 91/00360; WO 92/200373; and EP 03089. One method involves the separate preparation of antibodies having specificity for SEMA 5 and the effector molecule, respectively. Peptic F(ab′-gamma)₂ fragments from the two chosen antibodies are then generated, followed by reduction of each to provide separate Fab′gammaSH fragments. The SH groups on one of the two antibodies to be coupled are then alkylated with a cross-linking reagent, such as o-phenylenedimaleimide, to provide free maleimide groups on one antibody. This antibody may then be conjugated to the other by means of a thioether linkage, to give the desired F(ab′gamma)₂. Other approaches, such as cross-linking with SPDP or protein A may also be carried out.

Another method for producing bispecific antibodies is by the fusion of two hybridomas to form a quadroma. As used herein, the term “quadroma” is used to describe the productive fusion of two B cell hybridomas. In this method, two antibody producing hybridomas are fused to give daughter cells, and those cells that have maintained the expression of both sets of clonotype immunoglobulin genes (an anti-SEMA-5 immunoglobulin gene and an anti-effector immunoglobulin gene) are then selected. Following the isolation of the quadroma, the bispecific antibodies are purified away from other cell products. This may be accomplished by a variety of antibody isolation procedures, known to those skilled in the art of immunoglobulin purification (see, e.g., Antibodies: A Laboratory Manual, 1988). Protein A or protein G sepharose columns are preferred. See, also U.S. Pat. Nos. 5,855,866; 5,965,132; 6,004,555; 6,036,955 and 5,877,289, for example.

Therapeutic SEMA 5 Polypeptides

The invention also provides therapeutic SEMA 5 polypeptides. Such polypeptides can be used in vaccine compositions to enhance an immune response directed against cells overexpressing SEMA 5 polypeptides. In one aspect, a therapeutic vaccine composition according to the invention comprises an antigenic SEMA 5 polypeptide and a pharmaceutically acceptable carrier. In another aspect, the pharmaceutical composition comprises an immunogenic amount of a cell membrane which expresses a SEMA 5 polypeptide dispersed in a physiologically acceptable, nontoxic vehicle, which amount is effective to immunize a human against abnormal cellular proliferation (e.g., such as a cancer) associated with amplification and/or overexpression of a Sema 5 gene.

The invention also provides a method of vaccinating a subject against a SEMA 5 polypeptide comprising the step of inoculating the subject with a SEMA 5 protein or fragment thereof to elicit an immune response in the subject. In one aspect, the subject has a cancer associated with the overexpression of SEMA 5. In one aspect, a SEMA 5 fragment comprises about 9-20 amino acids of a SEMA 5 polypeptide, such as a SEMA 5C polypeptide (e.g., such as the polypeptide shown in FIG. 1), a SEMA 5B polypeptide, or a SEMA 5A polypeptide. Suitable antigenic portions of SEMA 5 polypeptides may be readily identified by synthesis of relevant epitopes, and analysis using methods routine in the art (see, e.g., Manca et al. Eur. J Immunol. 25:1217-1223 (1995); Sarobe, et al., J Acquir. Immune Defic. Syndr. 7: 635-40 (1994); and Shirai, et al., J Immunol. 152: 549-56 (1994), for example). Selection of the most appropriate portion of the SEMA 5 polypeptide for use as an antigen can be done by functional screening. Antigenicity may be measured by stimulation of antigen-specific MHC/HLA class I or MHC/HLA class II specific T cell line or clone. Alternatively, antigenicity may be determined by measurement of the ability to generate antibodies or T cells specific for the antigen in vivo as discussed further below.

In one aspect, epitopes are identified which are recognized by specific HLA haplotypes. Computer programs such as Motifscan can be used to scans a SEMA 5 polypeptide sequence for possible epitopes based on HLA binding motifs, while ELF (Epitope Location Finder Tool) may be used to identify potential CTL epitopes.

Fragments of at least 6 contiguous amino acids are useful in mapping B cell and T cell epitopes of a SEMA 5 polypeptide. See, e.g., Geysen et al., Proc. Nati. Acad Sci. USA 81: 3998-4002 (1984) and U.S. Pat. Nos. 4,708,871 and 5,595,915, for example.

In another aspect, the invention provides a method of producing activated immune cells which specifically recognize SEMA 5 polypeptides, comprising the step of exposing immune cells to an effective amount of an SEMA 5 polypeptide or fragment thereof, to activate the immune cells. Preferred immune cells include B cells, T cells, dendritic cells or other antigen presenting cells. In one aspect, antigen presenting cells are isolated from a subject prior to exposure to a SEMA 5 polypeptide or fragment thereof and then reintroduced into the subject subsequent to the exposure. In one aspect, the subject has a cancer associated with overexpression of SEMA 5.

Because of the low immunogenicity of peptides in general, SEMA 5 therapeutic peptides are typically administered with adjuvants to stimulate immune responses. Incomplete Freund's adjuvant and other oil-based adjuvants appear to be more effective and favor the induction of Th1 responses, while alum results in a preferentially Th2 response (Grun and Maurer, Cell Immunol. 121(1): 134-45 (1989)). Additional adjuvant approaches to enhance the response to peptides include the covalent association with lipopeptidic immunostimulants, or the encapsidation of peptides into liposomes (Kim, et al., Int J Oncol. 21(5: 973-9, 2002; Martinon, et al., J Immunol. 149(10): 3416-22, 1992). Certain cytokines also have been demonstrated to be useful (e.g., such as IL-2, IL-12 and the active fragment of IL-1 β).

Sema 5 nucleic acids encoding such peptides also may be used to provide SEMA 5 peptides for generating an immune response. Preferably, such nucleic acids are comprised within a viral vector comprising one or more inactivated virulence genes or comprising a restricted host range. Preferably, the vector is replication defective or incompetent or attenuated. Suitable vectors include, but are not limited to those based on (i.e., comprising portions of viral genomes of): poxviruses, adenoviruses, herpes viruses, alphaviruses, retroviruses, Epstein Barr viruses, lentiviruses, and picomaviruses. In one preferred aspect, the vector is a recombinant poxvirus vector. The recombinant nonvirulent poxvirus is preferably a vaccinia virus but may also be a fowlpox virus, such as a canarypox virus.

Preferably, the vector comprises one or more capsid polypeptides. In one aspect the one or more polypeptides are linked to a targeting molecule to facilitate selective infection of a cell (e.g., an antigen presenting cell, such as a dendritic cell, or a tumor cell). In one aspect, an effective amount of recombinant virus ranges from about 10 μl to about 25 μl of saline solution containing concentrations, preferably, of from about 1×10¹⁰ to 1×10¹¹ plaque forming units (pfu) virus/ml.

Administration of sources of SEMA 5 peptides or polypeptides to a subject results in the production of an expanded population of memory cells which are primed to produce a secondary response upon re-exposure to SEMA 5 antigens. This effect can be monitored by the ability of such cells to expand rapidly in the presence of SEMA 5 antigen presented by antigen presenting cells (APCs), or their ability to display a rapid antigen-specific cytolytic response even after the primary exposure to a SEMA 5 peptide/polypeptide.

The ability of vaccine compositions according to the invention to enhance a memory response can be evaluated by infecting mice with vaccine vectors according to the invention in the presence of a SEMA 5 antigen or a SEMA 5 antigen-encoding sequence. Mice are sacrificed at various days after antigen exposure and lymph node cells from naive mice, treated mice, or mice treated with buffer, are isolated and restimulated with vaccine antigen or an irrelevant antigen (e.g., such as hen egg lysozyme or an influenza antigen). Cell proliferation is monitored using methods routine in the art (e.g., by measuring the incorporation of ³H-thymidine. A significant increase in proliferation in memory cells as compared to buffer treated or naive mice (as determined using statistical methods well known in the art) is taken as an indication of an enhanced memory response. Preferably, vaccine compositions according to the invention are capable of producing an at least about 25%, at least about 50%, or at least about 100% increase in proliferation.

Additionally, or alternatively, cytolytic responses are monitored using methods routine in the art. Standard⁵¹-Chromium release assays are performed using HLA-matched or mismatched target B-lymphoblastoid cell lines labeled with ⁵¹-chromium (Amersham, Buckinghamshire, England) and pulsed with a pool of epitope peptides predicted to bind to the HLA molecule or a control peptide (e.g., such as an influenza antigen) at about 50 μM in multiple different wells of a microtiter plate. Individual peptides from a pool which reacts with the HLA molecule to stimulate a CTL response are then tested to identify the specific reactive peptide epitope. Chromium is counted in a scintillation counter (e.g., such as available at Wallac, Gaithersburg, Md.) and percent lysis calculated from the formula 100×(E-M/T-M), where E is the experimental release of chromium, M is release in the presence of medium without detergent (i.e., release which occurs because of a CTL response), and T is release in the presence of 5% Triton X-100 detergent. Results are regarded as positive if recognition of the SEMA 5 peptide is >10% above that of a control peptide in at least two separate assays).

Efficacy of a vaccine composition also can be evaluated by monitoring the level of a SEMA 5 antigen and/or the presence of antibodies in sera which specifically cross-react with SEMA 5.

SEMA 5Agonists or Antagonists

Variants of the SEMA 5 polypeptides that function as either SEMA agonists or as SEMA 5 antagonists can be identified by screening combinatorial libraries of mutants, e.g., truncation mutants, deletion, insertion, substitution mutations of a SEM 5 protein for agonist or antagonist activity. A library of SEMA 5 variants can be produced by, for example, enzymatically ligating a mixture of synthetic oligonucleotides into SEMA 5 coding sequences such that a degenerate set of potential agonists/antagonists is expressible as individual polypeptides, or alternatively, as a set of larger fusion proteins (e.g., for phage display) containing the set of SEMA 5 variant sequences.

There are a variety of methods that can be used to produce libraries of potential SEMA 5 variants from a degenerate oligonucleotide sequence. Chemical synthesis of a degenerate sequence can be performed in an automatic DNA synthesizer, and the synthetic sequence then can be ligated into an appropriate expression vector. Preferably, the library comprises in one mixture substantially all of the sequences encoding the desired set of potential SEMA 5 sequences. Methods for synthesizing degenerate oligonucleotides are known in the art (see, e.g., Narang Tetrahedron 39: 3 (1983); Itakura, et al., Annu Rev Biochem 53: 323 (1984); Itakura, et al., Science 198: 1056 (1984); Ike, et al. Nucl Acid Res 11: 477 (1983), for example).

In one embodiment, a library of coding sequence fragments can be generated by treating a double stranded PCR fragment of a SEMA 5 coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of a SEMA 5 protein.

In another aspect, recursive ensemble mutagenesis is used to enhance the frequency of functional mutants in the libraries (Arkin and Yourvan, Proc. Natl. Acad. Sci USA 89: 7811-7815 (1992); Delgrave, et al., Protein Engineering 6: 327-331 (1993)).

The functionality of variant SEMA 5 sequences can be tested by introducing them into a Sema 5 null background in Drosophila melanogaster in the presence or absence of a l(2)gl gene to identify sequences that suppress or restore a neoplastic phenotype. In one aspect, dominant suppressors of an l(2)gl neoplastic phenotype are selected for.

SEMA 5 Pharmaceutical Compositions

The SEMA 5 therapeutic nucleic acid molecules, antibodies and polypeptides can be incorporated into pharmaceutical compositions suitable for administration. Such compositions typically comprise the nucleic acid molecule, protein, or antibody (“the active agent) and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral, topical, transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). Preferably, the composition is sterile and pyrogen free.

The pharmaceutical carrier can be a solvent or dispersion medium containing water, ethanol, polyol (for example, glycerol, propylene glycol, and liquid polyethylene glycol, and the like), and suitable mixtures thereof. Antibacterial and antiflugal agents may be added, such as parabens, chlorobutanol, phenol, ascorbic acid, thimerosal, and the like. Isotonic agents may be included such as sugars, polyalcohols such as manitol, sorbitol, and sodium chloride.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those described above. Active ingredients may be vacuum dried or freeze-dried as is known in the art.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash. Pharmaceutically compatible binding agents, and/or adjuvant materials can be included as part of the composition.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No.4,522,811, for example.

Dosages are dictated by and directly dependent on the unique characteristics of the active compound and the particular therapeutic effect to be achieved.

The nucleic acid molecules of the invention can be inserted into vectors for gene therapy. Gene therapy vectors can be delivered to a subject by, for example, intravenous injection, local administration (see e.g., U.S. Pat. No. 5,328,470) or by stereotactic injection (see e.g., Chen et al. Proc. Natl. Acad. Sci USA 91: 3054-3057 (1994)). A pharmaceutical preparation comprising a gene therapy vector can include the gene therapy vector in an acceptable diluent, or can comprise a slow release matrix in which the gene delivery vehicle is imbedded. Alternatively, where the gene delivery vector comprises a viral vector (e.g., a retrovirus, adenovirus, etc.) can be produced intact from recombinant cells, and the pharmaceutical composition can include one or more cells that provide the virus. Alternatively, viral particles comprising a nucleic acid molecule of the invention can be provided in a suitable pharmaceutical carrier.

The pharmaceutical compositions can be included in a container, pack, or dispenser together with instructions for administration.

Kits

The invention further provides kits for facilitating the assays and methods described herein. In one aspect, a kit comprises a molecular probe for specifically binding to a Sema 5 gene or gene product (e.g., RNA or protein) and reagents for detecting reaction complexes formed between the molecular probe and the Sema 5 gene and/or gene product. In another aspect, the kit comprises a sample comprising amounts of a Sema 5 gene product characteristic of a normally proliferating cell. The sample may comprise a cell, cell lysate, tissue section, and the like. In another aspect, the kit comprises a sample comprising amounts of a Sema 5 gene product characteristic of an abnormally proliferating cell. For example, the sample may comprise a cell, cell lysate or tissue section from a tumor. In a further aspect, the kit comprises a tissue or cell samples of both normally and abnormally proliferating cells in multi-well plates or on a substrate (e.g., such as a glass slide) for simultaneous detection of Sema 5 in normal and abnormally proliferating cells. Preferably, simultaneous detection is done concurrently with testing of a test sample from a subject being tested for overexpression of a Sema 5 gene product.

Identification of Modulators of SEMA 5 Polypeptides

The invention further provides a whole-organism based assay for identifying modulators of SEMA 5. Preferably, the whole organism is small and multicellular with a rapid generation time and comprises multiple germ layers. More preferably, the organism comprises a high degree of conservation of the various signaling pathways involved in the etiology of human disease; can be grown rapidly in large numbers and comprises genetically mapped marker genes to facilitate mapping of newly identified mutations.

In particular, the invention provides an HTS system for identifying genes which modulate SEMA 5 and/or an interaction between a mutated Sema 5c gene and a mutated /1(2)gl gene. The system exploits the rapid growth and well-characterized genetics of Drosophila melanogaster.

The high degree of conservation of morphogenetic processes between Drosophila and humans makes Drosophila a powerful system to use to screen, identify and characterize molecules that are functionally required for cellular invasion during cancer and metastasis. The components of signaling pathways between Drosophila and humans are also highly conserved.

In one aspect, an HTS method according to the invention comprises introducing a tissue expressing a reporter sequence in an adult fruit fly. Preferably, the tissue is derived from a fly comprising a mutated gene of l(2)gl which mutation results in non-tissue specific abnormal cell proliferation. The tissue further comprises a mutation of the Sema 5c gene which suppresses the neoplastic phenotype typically caused by the presence of the l(2)gl mutation. In one aspect, the l(2)gl mutation is a functional null mutation (e.g., a deletion or a mutation which otherwise eliminates activity of the l(2)gl gene product). However, other types of mutations identified which result in a neoplastic phenotype are also encompassed within the scope of the invention. Such mutations include hypomorphic mutations, neomorphic mutations, and conditional mutations (e.g., temperature sensitive mutations) and the like. The Sema 5c mutation may be a functional null mutation, a hypomorphic mutation, neomorphic mutation, conditional mutation, and the like. In another aspect, a mutated non-Drosophila Sema 5 gene is introduced into a fly (e.g., in a background null for the fly Sema 5c gene)

The tissue to be introduced into the adult fly further comprises a candidate mutated modulator gene that is capable of modulating the suppressed neoplastic phenotype in the adult fly that arises because of the interaction between the mutated l(2)gl and Sema 5c gene. For example, the modulator gene can modulate tumor induction (e.g., tumorgenicity, or numbers of tumors), tumor growth (e.g., numbers of cells in a tumor or tumor size) and/or metastasis (invasion into different tissues). In another aspect, the adult fly comprises the mutated modulator gene and the tissue being introduced into the adult fly is wild type at the modulator gene locus. The modulator gene may be a gene which is neither l(2)gl or Sema 5 or may be a second site mutation in the mutated Sema-5c gene.

The presence or expression of the reporter sequence in cells from a plurality of different tissues in the adult fly is evaluated and one or more of: a change in the numbers of different tissues expressing the reporter sequence and a change in the quantity of the reporter sequence, in one or more tissues, identifies the presence of one or more mutated genes in the adult fly which are functional modulators of SEMA 5. As used herein monitoring the expression of a reporter sequence in “cells from a plurality of different tissues” refers to monitoring the presence of and/or expression of the reporter sequence and/or monitoring the activity of a reporter sequence product (e.g., such as a protein or transcript). The cells do not need to be isolated from the fly and can be monitored in situ. The term “plurality” refers to at least two.

Preferably, the reporter sequence is operably linked to a transcriptional regulatory element that is capable of driving expression of the reporter sequence in transplanted neoplastic cells. The product of the reporter sequence may be visually detectable, either in a fluorescence assay or after interacting, directly or indirectly, with a chromogenic substrate. Examples of such reporters include, the lacZ protein (β-galactosidase), green fluorescent protein (GFP), alkaline phosphatase, horseradish peroxidase, blue fluorescent protein (BFP), and luciferase photoproteins such as aequorin, obelin, mnemiopsin, and berovin (see, e.g., U.S. Pat. No. 6,087,476).

However, a reporter sequence may also be any nucleic acid sequence that is not found in the host fly and which may be detectable by a suitable assay (e.g., such as by PCR). Similarly, a reporter sequence can encode an antigenic sequence (e.g., a peptide) not typically expressed in the host cell, allowing neoplastic cells to be recognized by using antibodies to detect expression of the antigenic sequence. Commonly used and commercially available epitope tags include sequences derived from, e.g., influenza virus hemagglutinin (HA), Simian Virus 5 (V5), polyhistidine (6xHis), c-myc, lacZ, GST, and the like. Antibodies specific to these epitope tags are generally commercially available. The expressed reporter can be detected using an epitope-specific antibody in an immunoassay or by FACs analysis.

Examples of suitable transcriptional regulatory elements include the Alcohol dehydrogenase (ADH) gene promoter, hsp 70 promoter, hsp 82 promoter, and the like. Reporter sequences can be integrated into the Drosophila genome using methods known in the art, such as P-element transformation, using the presence of a marker gene to follow the inheritance of the P-element. Suitable marker genes include white and rosy which affect eye color. Other marker genes in Drosophila include, but are not limited to, yellow, ebony, singed, and Mwh, which are body color or morphology markers. A comprehensive list of markers for Drosophila may be found in Ashburner (In D. melanogaster: A Laboratory Manual, (1989) Cold Spring Harbor, N.Y., Cold Spring Harbor Laboratory Press: pp. 299-418).

Preferably, mutations in candidate modulator genes are generated at random, allowing the entire genome to be scanned for potential modulator genes. More preferably, mutations are generated using P-elements comprising markers that can be used to select for viable homozygotes bearing two copies of a mutated gene. The proliferation of l(2)gl-/l(2)gl-::sema-5c-/sema-5c- cells in such flies can be tracked by assaying various cells, tissues, or body segments, of the adult fly for the expression of the reporter gene expressed by the neoplastic cells. In one aspect, the P-element comprises both the marker gene and the reporter sequence. This assay allows for quantitative and qualitative measures of abnormal cell proliferation in the flies being screened.

P-elements comprise sequences recognizable by a transposase that enables the P-elements to be inserted into or removed from the genome. In fly strains expressing repressors of the transposase, the P-elements do not excise and are stably integrated in a fly's genome. When crossed to a fly strain lacking such repressors, P-elements will “hop” and insert at different genomic locations and can disrupt gene function when they land in a gene. By crossing back to a strain that comprises repressors, the newly inserted P-elements will be stable at their new locations. Transposition is predominantly limited to the germline and so the insertions are heritable. Therefore, P-elements can be used to randomly mutagenize the Drosophila genome, producing stable, heritable mutations.

Mutated genes can be readily cloned using the P-elements as tags for these genes. Mapping is simplified by the well-developed cytogenetic and molecular analyses permitted by Drosophila. The functional role of the gene can be verified using P-element mediated rescue to introduce wild-type copies of the gene back into the fly and/or to monitor the effect of excision of P-elements from a particular gene.

In one preferred aspect, the same P-elements that are used to randomly mutagenize the genome also carry the reporter sequence. Preferably, the P-elements also comprise a marker gene allowing the inheritance of the P-elements to be correlated with the expression of the marker gene.

Also, preferably, the P-element being used as an insertable element does not itself encode transposase. For example, transposase function may be provided by an integrated P-element (e.g., such as the transposase source, P(ry⁺Δ2-3) which is itself unable to hop from the genome or by a crippled P-element vector which is co-introduced with the mutagenizing P-element.

A DNA construct comprising a P-element, and preferably comprising a reporter sequence and marker gene, is injected into embryos of M strain females which lack P-elements and which do not express the marker gene. Suitable marker genes include those which provide a visible, easily selectable phenotype such as eye color, body color, wing morphology, and the like, as discussed above. In one aspect, the P-element comprises a mini-white gene whose expression in flies bearing the white mutation restores a red eye color to otherwise white-eyed flies. Suitable P-element vectors are described in, Pirrotta, et al. Vectors: A Survey of molecular Cloning Vectors and Their Uses, edited by R. L. Rodriguez and D. T. Denhardt, Butterworths, Boston, 1988; and Rubin and Spradling, Nucleic Acids Res. 11(18): 6341-51 (1983), for example. In some aspects, enhancer or promoter trap vectors are used. For example, the P-element construct can comprise a promoter-less reporter gene sequence. Expression of the reporter gene sequence will only occur when the P-element construct is integrated downstream of a promoter and expression of the reporter gene will therefore reflect the transcription pattern of the modulator gene. Because the marker gene comprises a promoter, all insertion events will be detectable, not just the ones which bring the reporter gene in suitable proximity to the marker gene promoter. See, e.g., as described in Lucasovich, et al., Genetics 157: 727-742, (2001).

Microinjection is carried out using methods known in the art, such as described in Van Deusen, J Embry. Exp. Morph. 37: 173 (1976). Typically, embryos are collected on lightly yeasted agar plates for one hour, then transferred to 17-18° C. Chorions are removed and embryos are aligned on double stick tape. Preferably, embryos are covered in oil (e.g., fluorocarbon oil) to minimize drying. Injections are performed at the posterior end of the embryo, since this end comprises the developing germ line cells of the fly.

After injection, embryos are maintained in a humidified chamber at 17-18° C. Hatched larvae are removed from the oil and placed on standard Drosophila cornmeal-molasses-yeast medium with subsequent development at 21-23° C.

Surviving embryos that develop into fertile adult flies are mated to non-M strains which also lack the marker gene. Progeny are examined to identify those flies that express the marker gene and therefore which include the P-element. Of these flies, a subset are crossed to flies bearing balancer chromosomes to prevent chromosomes bearing the P-element from recombining, to maintain stocks of flies bearing the mutant modulator genes, and to otherwise facilitate mapping of the P-element. Another subset is mated to other progeny in the subset to generate flies that are homozygous for the P-element.

Alternatively, M strain females are simply mated to males comprising a mutagenic P-element in their genome and expressing the Δ2-3 element, i.e., “jump-start” males.

Preferably, mutations are selected which result in the production of viable adult flies when homozygous for the P-element.

In yet another embodiment, flies from a stock center comprising P-element insertions may be crossed to by appropriate breeding schemes to produce flies that are homozygous for the P-element insertion and l(2)gl and Sema 5c mutations. l(2)gl- flies can be obtained from the Berkley Drosophila Genome Project (BDGP) Gene Disruption Project are available from the Bloomington Stock Center (Bloomington, Ind.) (see, e.g., Spradling, et al., Genetics 153: 135-177, (1999)). Sema 5c mutant flies can readily be generated using the methods described herein.

The use of P-elements in Drosophila is well-known in the art and is described in, for example, Rubin and Spradling, Science 218: 348-53 (1982); U.S. Pat. No. 4,670,388; Engels, Cold Spring Harbor Symp. 45: 561 (1981).

Methods of fly husbandry are also routine in the art and described in, for example, in Ashburner, Fly Pushing: The Theory and Practice of D. melanogaster Genetics, Cold Spring Harbor Press, Plainview N.Y. (1977).

Identification of Modulator Mutations

Flies are bred which are homozygous both for the modulator mutation and the l(2)gl and Sema 5 mutations and grown to larval stages using techniques well known in the art. See, Ashburner, 1977, supra. Cells from brain or imaginal discs are isolated for transplantation into adult flies that are wild type for both the modulator gene and tumorigenic gene and which do not express the reporter sequence. Cells or tissue fragments are then injected into the abdomens of female adult flies. Samples from greater than 100,000 different mutant lines may be examined in this way. Neoplastic tissues from flies homozygous for the mutated l(2)gl gene, from l(2)gl-/l(2)gl::Sema 5c-/Sema5c- flies, from Sema 5c-/Sema5c- flies, and/or from flies which are wild-type for l(2)gl and Sema 5c and modulator gene is used as a control. Preferably, except for differences at l(2)gl and Sema 5c and modulator gene, the flies are otherwise genetically identical.

Alternatively, the host flies may be screened for modulator genes which affect the neoplastic phenotype of l(2)gl-/l(2)gl::Sema 5c-/Sema5c- tissues, by mutagenizing a non-l(2)gl background (e.g., with P-elements) and selecting for viable homozygous flies in which the establishment or metastasis of Sema 5c/l(2)gl neoplastic cells is altered, i.e., by transplanting cells from l(2)gl-Il(2)gl::Sema 5c-/Sema5c- larvae into adult flies homozygous for the modulator mutation. Such an assay may be used to screen for altered cell membrane receptors, extracellular matrix proteins and the like, that may be involved in the establishment or invasion of cancerous cells.

The tumorigenic and metastatic potential of these transplanted cells is evaluated by monitoring the expression of the reporter sequence in a plurality of cells in the adult fly. The assay used will generally depend on the nature of the reporter sequence selected. Preferably, the assay is one that can be performed in less than a day, and more preferably, can be performed in a few hours. Methods of detecting reporter gene expression in Drosophila are well known in the art. For example, Brandes, et al., describes detecting luciferase expression in Neuron 16: 687-692; Chalfie, et al., Science 263: 802-805

The plurality of cells is isolated from a variety of tissues types and/or body segments so that the impact of the modulator gene on cellular proliferation in the entire organism can be determined. Both tumorigenesis (i.e., numbers of flies with tumors in a population of flies; tumor size in an individual fly) and metastasis (number of tumors per fly and/or numbers of body segments/tissue types affected) can be monitored and quantified. In one aspect, cells from one or more of: the abdomen, thorax, head, wing and leg are obtained and the expression of the reporter sequence is determined and quantitated. In another aspect, whole body sections are isolated for immunohistochemistry or in situ hybridization analysis of reporter gene expression. Whole body immunohistochemistry may also be performed (i.e., without sectioning). A change in the numbers of different tissues expressing the marker gene and a change in the quantity of the marker gene product, in one or more tissues, identifies the presence of one or more mutated genes in the adult fly which are functional modulators of the neoplastic phenotype.

Cloning of Associated Genes

Transposon-mediated mutagenesis such as mediated by P-elements, provides a useful way to map and clone modulator genes. P-element and/or reporter sequences can be used as probes in hybridization assays to cytogenetically map the site of the modulator mutation to a polytene chromosome band. For example, chromosomes can be prepared from larval salivary glands and hybridized in situ with a labeled probe. See, e.g., as described in Spradling Cell 27: 193 (1981).

The marker gene can be used in standard genetic assays (i.e., crosses) to map the modulator gene identified by P-element insertion. P-element sequences can be used to amplify sequences flanking an insertion site. For example, PCR can be performed using as primers, one or more of P-element sequences, the reporter sequence, and the marker sequence. See, e.g., Allen, et al., PCR Methods Appl. 4: 71-75. Amplified sequences flanking the P-element sequences can be sequenced using methods routine in the art and sequence information can be used to query a database of Drosophila sequences and/or sequences of other organisms (e.g., such as human beings).

Alternatively, or additionally, P-element sequences, reporter sequences, marker sequences, and/or amplified sequences may be used as hybridization probes to isolate genomic or cDNA clones from libraries derived from flies carrying the mutated modulator gene. Clones can be validated by cytogenetic analysis and/or mapping crosses. As an additional validation step, the ability of a clone to rescue the mutant modulator phenotype can be determined. In one aspect, wild type modulator gene sequences are cloned into P-element vectors, and the ability of the sequences to rescue the modulator mutant phenotype is determined. Such vectors also provide the opportunity to increase the dose of the modulator gene product and to evaluate the affect of dosage on the neoplastic phenotype.

Preferably, cloned sequences are used to identify homologous sequences in human beings. For example, nucleic acid and protein sequences modulator genes can further be used as query sequences to perform a search against sequence databases to, for example, identify other family members or related sequences. Such searches can be performed using the NBLAST and XBLAST programs (version 2.0) of Altschul, et al. (J. Mol. Biol. 215: 403-10, 1990). BLAST nucleotide searches can be performed with the NBLAST program, with exemplary scores=100, and wordlengths=12 to obtain nucleotide sequences homologous to or with sufficient percent identity to the nucleic acid molecules of the invention. BLAST protein searches can be performed with the XBLAST program, with exemplary scores=50 and wordlengths=3 to obtain amino acid sequences sufficiently homologous to or with sufficient % identity (e.g., preferably, at least 60% identity). To obtain gapped alignments for comparison purposes, gapped BLAST can be used as described in Altschul, et al. (Nucleic Acids Res. 25(17): 3389-3402 (1997)). When using BLAST and gapped BLAST programs, the default parameters of the respective programs (e.g., XBLAST and NBLAST) can be used.

The biological role of a cloned modulator gene is evaluated is methods known in the art. In one aspect, the expression of the gene is determined (e.g., by Northern, dot blotting, RT-PCR, in situ hybridization, immunoassays, and the like). In another aspect, the interaction of the modulator gene with one or more members of a molecular pathway is determined. Preferably, the molecular pathway is a signaling pathway. For example, in one preferred embodiment, larval brain extracts from flies homozygous for the modulator mutation and wild type or homo- or heterozygous for the l(2)gl and Sema 5c mutation are arrayed onto a suitable substrate (e.g., such as a nitrocellulose slide) and the expression of a plurality of different pathway molecules in these extracts is determined using antibodies to modified (e.g., phosphorylated) and/or unmodified pathway molecules. Suitable pathway molecules whose expression can be evaluated include, but are not limited to: expression products of PI3K; T-ERK; SMAD; P-SAMD1; Akt; Mad; cleaved caspase 3; Decapentaplegic (Dpp); l(2)gl genes, and the like. Arrays of nucleic acid samples can similarly be evaluated to monitor gene expression (e.g., in RT-PCR assays).

Control samples may also be included in the array, such as samples from wild type flies and/or samples from organisms that are not flies (e.g. such as plant cell samples, and the like). Combinations of samples such as described above may be included in the arrays and variations of these arrays are obvious and are encompassed within the scope of the invention.

Identification of Modulator Compounds

In a further aspect, a method according to the invention comprises obtaining tissue obtained from larvae of a fly homozygous for l(2)gl-/l(2)gl::Sema 5c-/Sema5c- and introducing cells or tissue into a wild type adult fly. Alternatively, the tissue is obtained from a fly that overexpresses Sema 5, which overexpression causes the tissue to be tumorigenic. A fly overexpressing Sema 5 can be generated by introducing multiple copies of a Sema 5 cDNA into the fly by P-element mediated transformation.

A candidate modulator compound is introduced into the nutrient medium on which an adult fly (preferably, newly eclosed), or a larval form, feeds. The ability of the modulator to alter the pattern of tumor growth in the fly is assessed. Populations of flies (e.g., greater than 100,000) can be screened in this way to identify candidate agents that affect tumor growth. The proliferation of neoplastic cells can be tracked by monitoring the expression of a reporter sequence inserted into the genome of such cells and assaying various segments of the adult fly for the presence of, levels of, and/or activity of, the reporter. This assay allows for quantitative and qualitative measures of abnormal cell proliferation in the flies being screened.

As above, both tumorigenesis and metastasis can be monitored and quantified. In one aspect, cells from one or more of: the abdomen, thorax, head, wing and leg are obtained and the expression of the reporter sequence is determined and quantitated. In another aspect, whole body sections are isolated for immunohistochemistry or in situ hybridization analysis of reporter gene expression. Alternatively, whole mounts can be evaluated. A change in the numbers of different tissues expressing the marker gene and a change in the quantity of the marker gene product, in one or more tissues, identifies the presence of one or more candidate modulator compounds in the adult fly which are functional modulators of the neoplastic phenotype.

Alternatively, one or more cells comprising a neoplastic phenotype may be transplanted into adult flies that have been fed, and/or are being fed different compounds to be assayed. The effect of the compounds on the induction and invasion of tumors is monitored generally as described above. In yet another aspect, the one or more cells are transplanted into adult flies and the adult flies are exposed to compound after transplantation.

These types of HTS assays also allow for a determination of the general toxicity of modulator compounds through 50% lethal dose (LD₅₀) computations.

Because large numbers of compounds can be screened, in one aspect, the method comprises screening a compound library for a modulator of the tumorigenic gene. Compound libraries may be purchased commercially (e.g., such as LeadQuest™-libraries from Tripos (St. Louis, Mo.)) or may be synthesized using methods well known in the art. Compounds may be introduced into the nutrient media on which larvae or adult Drosophila feed and the affect of the compounds can be assayed for by performing the whole-organism based-screening assay described above. Compounds may be delivered to individual flies or to groups of flies.

Suitable compound which can be tested, include, but are not limited to, carbohydrates, polyalcohols (e. g., ethylene glycol and glycerol), polyphenols (e.g., hydroquinones and tetracylines), small molecules, drugs, proteins, peptides, or pharmacophores thereof, peptoids, peptidomimetics, nucleic acids, nucleosides, metabolites, nucleic acid aptamers, protein aptamers, and the like. Compounds may be based on (i.e., pharmacophores of) naturally occurring extracellular or intracellular signaling molecules or their derivatives or the like. Compounds may be provided in a delivery vehicle such as a sucrose solution or in a liposome formulation.

In one aspect, eggs of the suitable genotype are collected on a nylon mesh and placed onto standard fly food. Approximately three to five day old larvae (third larval instar) are then collected and placed in suitable containers such as multiwell culture dishes comprising wells with a nutrient layer (e.g., such as agar supplemented with yeast) or in individual culture dishes. Compounds are either present in, or added to, the nutrient layer. Compounds may be provided to different larvae or sets of larvae at different doses. Delivery of compounds can be automated using an automated injection robot. Individual containers for larvae and or flies may be tagged using means known in the art such as bar code labels or radiofrequency tags.

Following a suitable exposure period, one or more cells from each larva (or sets of larvae) exposed to a particular compound are obtained and introduced into an adult fly to evaluate the neoplastic potential of the cells. Cells from different tissues are evaluated to survey the organism, e.g., samples can be obtained from the head, thorax, abdomen, leg, etc. to survey the expression of the reporter sequence. In one aspect, the level of reporter gene expression and/or spread of reporter gene expression is monitored. Where multiple flies are used to test particular compounds, tumor incidence in a plurality of flies can be determined. In still another aspect, the effect of different doses of compounds can be evaluated.

The HTS assay system is used to identify modulator compounds that affect the neoplastic phenotype. However, the system may also be used to assay determine the carcinogenic potential of known or unknown compounds. Over 100,000 compounds may be screened in a short period in the HTS format described above. Because the biological affect of the compound on the entire organism is evaluated, more biologically relevant compounds should be identified than in cell-based screening assays.

As additional mutated alleles of l(2)gl and/or Sema 5 are identified, dominant interactions at between the loci may be identified, i.e., the above screens may be modified to provide tissue which is heterozygous for one or both of the loci to measure neoplasia and suppression thereof. Such modified screens are encompassed within the scope of the instant invention.

### EXAMPLES

The invention will now be further illustrated with reference to the following examples. It will be appreciated that what follows is by way of example only and that modifications to detail may be made while still falling within the scope of the invention.

### Example 1

Flies were reared in shell vials on standard corrneal, molasses, and yeast medium at 20° C. Second chromosome lethal mutations were maintained over balancers marked with y⁺ and CyO mutations in stocks that were homozygous for the y mutation on the X-chromosome. Mutant larvae could be identified on the basis of expression of the y mutant phenotype.

Generation of Homozygous Mutations that Disrupt Metastasis

P-element insertion mutations were generated in a l(2)gl heterozygous background. A Plac W P-element inserted on the X chromosome was randomly mobilized in a heterozygous lethal giant larvae background by combination with the ‘jumpstarter’ P-element strain P(ry+; Δ02-3). Autosomal insertions were mapped by standard genetic methods using a yw/yw;+/+;+/+ stock and examining the segregation of CyO and the w⁺ marker. A homozygous P-element stock was established from each independent insertion.

Homozygous l(2)gl larvae were isolated from P-element lines carrying two copies of the P-element insertion (FIG. 1A). Over 124,000 flies heterozygous for l(2)gl were screened for transposition of a single P-element originally on the X-chromosome. The mini-white gene was used as a marker to follow inheritance of the P-element via eye color. Nine hundred and eighty-six P-element insertions were isolated and mapped in this way. A line was established for each insertion carrying both copies of the P-element. In some cases, homozygosity of the P-element caused embryonic or early larval lethality. Mutations were selected which were homozygous viable.

Brain lobes from armadillo-lacZ marked larvae; were dissected and cut into halves. Each fragment was injected into the abdomen of a βgal^{ml} (a mutant that lacks endogenous 6-galactosidase expression) adult female using a 33 gauge needle, where it was cultured for 21 days at room temperature. Hosts that had received transplants were opened along the ventral midline and fixed in 3.7% formaldehyde in phosphate buffered saline (PBS). β-galactosidase in the donor tissue was detected by staining overnight at 37° C. in 0.02% X-gal in 10 mM Na pyrophosphate, 0.15 M NaCl, 1.0 MM MgCl₂, 5 mM ferricyanide, and 5 mM K ferrocyanide (Specialty Media). Secondary tumors were defined as β-galactosidase-marked cells distinct from the :implanted tumor, which was defined as the primary tumor.

When l(2)gl brain fragments were injected into adult hosts (see, arrowhead in FIG. 1B), the injected tissue proliferated as a primary tumor (T) and invaded adjacent tissue. Cells migrated away from the primary tumor to generate widespread metastatic colonies (M) (FIG. 1B-D). As previously described for the l(2)gl phenotype (Woodhouse, et al., 1998, supra), metastatic colonies are found in the abdomen (57%), thorax (70%), head (39%), wing (35%) and leg (48%). FIG. 1D shows a tissue section of invasive l(2)gl/l(2)gl tumors in host thorax muscle.

Homozygous P-element induced mutations were identified in which this phenotype was disrupted. Excision of the P-element was shown to restore the neoplastic phenotype. Tumorigenic and metastatic cells were visualized by lacZ staining after 21 days. FIG. 1E shows the neoplastic phenotype of the 97-2 insertion l(2)gl/ 97-2 insertion l(2)gl line. A primary tumor (T) is observed but no metastasis. FIG. 1F shows reversion back to a neoplastic phenotype in 97-2 excision l(2)gl/97-2 excision l(2)gl flies. FIG. 1G shows suppression of the neoplastic phenotype in the l(2)gl/l(2)gl; 23-2 insertion/23-2 insertion line. Tumorigenesis and metastasis is suppressed. FIG. 1H shows reversion to a metastatic phenotype in l(2)gl/l(2)gl; 23-2 excision/23-2 excision flies.

Identification of Functional Mutations and Cloning of Associated Genes

Insertion 97-2 completely blocked metastasis although it did not inhibit primary tumor growth (FIG. 1E) (12/12 in each group). Excision of the P-element reverted this line to the full l(2)gl metastatic phenotype (12/12) (FIG. 1F). P-element insertion 115-1 accelerated the lethality of injected tumors (12/12 in each group). When l(2)gl tissue was transplanted into 12 hosts, one half of the hosts survived 36 days, compared to 24 days for 115-1/l(2)gl flies. Furthermore, all of the hosts injected with l(2)gl tissue died within 60 days compared to 42 days for 115-1/l(2)gl (P<0.01).

A third P-element insertion, line 23-2, disrupted both the tumorigenesis and metastasis pattern of l(2)gl brain tissue (12/12 in each group). Two copies of this P-element insertion completely blocked proliferation of the l(2)gl primary tumor (FIG. 1G) but did not alter viability of the larva or grossly modify l(2)gl brains, which are composed of overgrown tissues with loosely adherent cells. Excision of the P-element in line 23-2 resulted in reversion to a tumorigenic and metastatic phenotype (12/12) (FIG. 1H) (p<0.01). Thus, the gene disrupted in this line is required for the l(2)gl malignant phenotype.

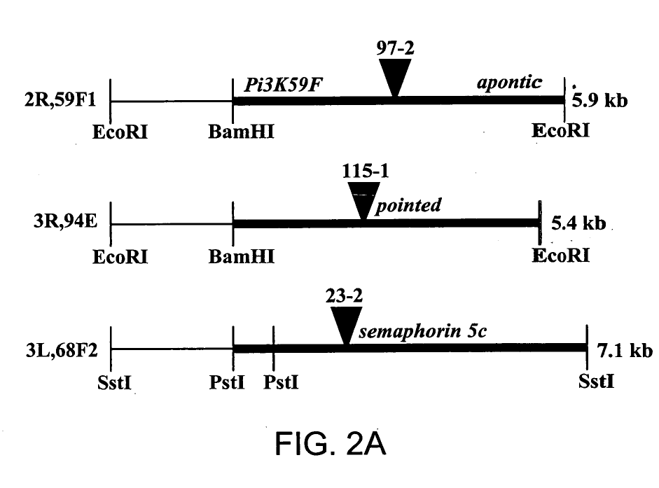

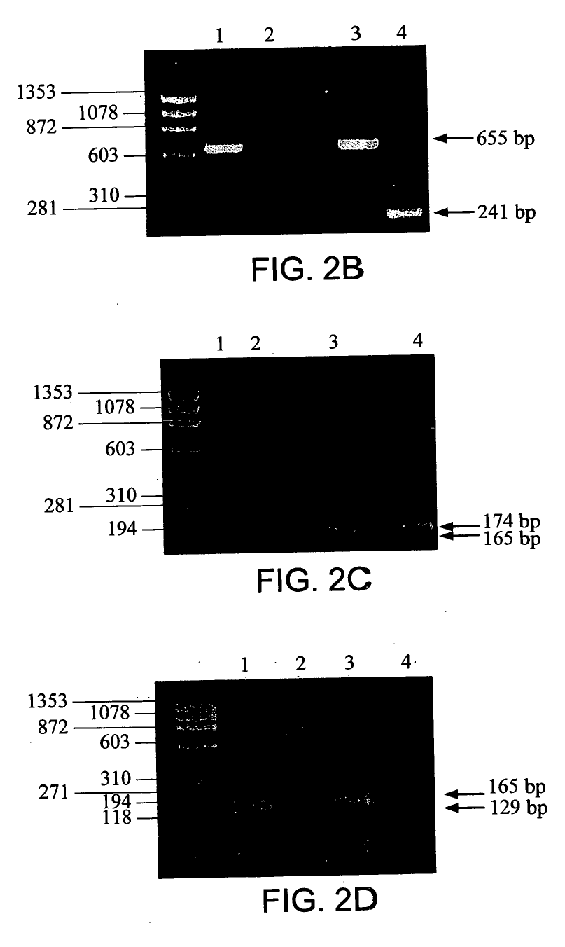

The genomic DNA at the 3′ end of the P-element was isolated by plasmid rescue (FIG. 2A) from adult Drosophila from each P-element line. The DNA was cut with a restriction enzyme and phenol-chloroform extracted. An EcoRI genomic fragment was isolated from lines 97-2 and 115-1 and an SstI genomic fragment was isolated from line 23-2. The fragments were ligated and phenol-chloroform extracted. One shot TOP 10 (Invitrogen) cells were transformed with the ligation mix. DNA was extracted from individual colonies and analyzed by restriction mapping using the second polylinker sites (BamHI for lines 97-2 and 115-1 and PstI for line 23-2). Cloned flanking sequences were sequenced at the NIH DNA minicore facility. Random hexamer-based reverse transcription was performed from third instar larvae total RNA.

The 97-2 insertion is on the right arm of the second chromosome at 68F2, between the Pi3K59F and apontic genes. The 115-1 insertion is on chromosome 3 at 94E in the pointed gene. The 23-2 P-element is inserted on the left arm of chromosome 3 at 68172 in the sema-5c gene. Confirmation of this localization was performed by PCR amplification of genomic DNA from each line with specific primers. One primer matched the P-element sequence near the 3′ end and the second primer matched a sequence in the flanking genomic DNA. PCR amplification with each insertion/P-element primer pair resulted in a product of a predicted size for that P-element line, but did not amplify a product in other lines including the parental line (see, e.g., FIG. 2B). PCR conditions were: 1 cycle 94° C. for 5 minutes, 35 cycles of 45 seconds at 94-C, 45 seconds at 58° C., 45 seconds 72° C., 1 cycle at 72° C.

P-Element Insertions Caused Up-Regulation of Apontic and Pointed

Expression of the apontic and pointed genes were examined by RT-PCR in lines 97-2 and 115-1. The expression of apontic is present in the P-element line 97-2 and absent in the parental line (FIG. 2C). P13K was examined in the 97-2 line, as the insertion is between the Pi3K59F and apontic genes and could affect either or both genes. The protein expression levels of P13K in larval brains from the 97-2 insertion were not significantly altered. Thus, the inhibitory effect caused by the P-element insertion in line 97-2 on metastasis is due to the expression of apontic. The pointed gene was strongly up-regulated in the 115-1 insertion line compared to the E1 parental line (FIG. 2C). This caused increased host lethality of l(2)gl/l(2)gl,l 15-1/115-1 compared to l(2)gl/l(2)gl flies.

P-Element Insertion Causes Loss of SEMA5C Protein Expression

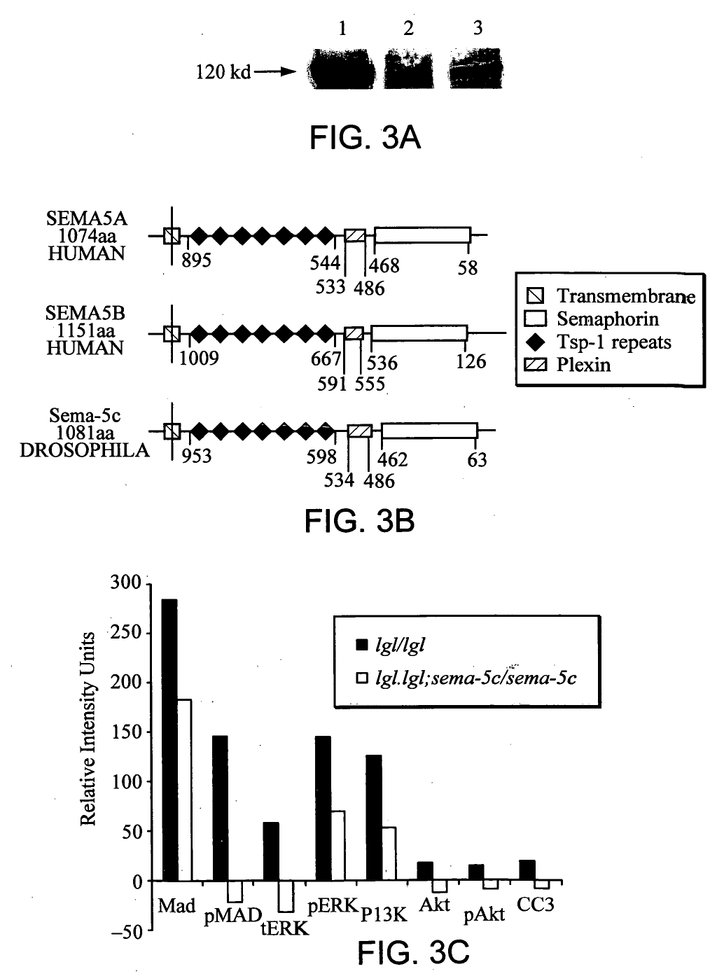

SEMA5C was undetectable in protein extracts from dissected brain tissue of homozygous 23-2 flies. Excision of the P-element resulted in recovery of protein expression (FIG. 3A) and restoration of the malignant phenotype (FIG. 1H). Based on sequence homology, two related mammalian semaphorins were identified with sequence domains similar to those of SEMA5C, SEMA5A and SEMA5B. All are class 5 semaphorins, containing thrombospondin repeats, a sema domain and a transmembrane domain (FIG. 3B).

Up-Regulation of Murine and Human SEMA5A in Metastatic Cell Lines

The expression level of SEMA5A was studied in cell lines of varying metastatic potential. Larval brain extracts were prepared by dissection of brains from late third instar larvae and homogenization in RIPA buffer containing 500 μM AEBSF hydrochloride, 150 mM aprotinin, 1 μM E-64, 0.5 mM EDTA disodium, 1 μM leuptin hemisulfate. 2X Tris-Glycine. SDS sample buffer (Novex) with 4% β-mercaptoethanol was added and extracts were boiled 5 minutes. Cell line lysates were prepared in 25 μM HEPES, pH7.5, 150 MM NaCl, 1% Igepal CA-630, 10 mM MgCl₂, 1 mM EDTA, 2% glycerol, 500 μM AEBSF hydrochloride, 150 mM aprotinin, 1 μM E-64, 0.5 mM EDTA disodium, 1 μM leuptin hemisulfate.

Anti-peptide antibodies were generated and affinity purified against the sequence SVRIGLPKEESRN (SEQ ID NO. 1) in the plexin domain of the SEMA5C protein. Primary antibodies used were anti-P-SMAD1 (Cell Signaling) and anti-Tubulin 1:2000 (Sigma) antibodies. Binding was detected using ECL (Amersham) as is known in the art. To perform immunofluorescence, MDA435 cells were fixed in tissue culture dishes with 4% neutral buffered formaldehyde for 30 minutes. Stained cells were mounted in aqueous mounting media (DAKO).

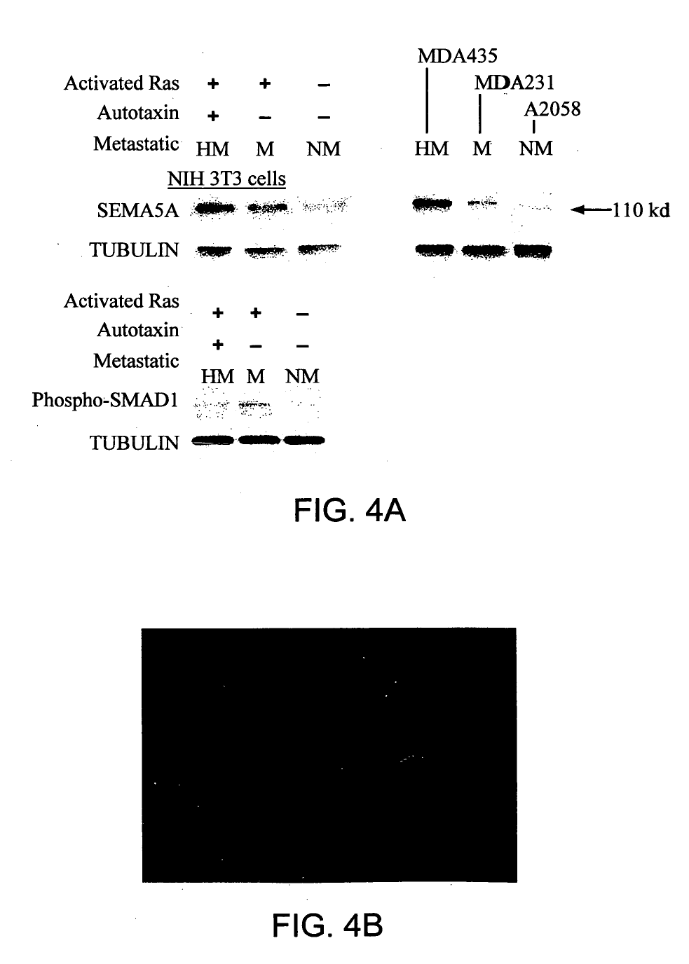

Western blot analysis using the anti-semaphorin antibody generated against the Drosophila SEMA5C revealed a single cross-reacting protein in murine as well as human cell lysates that corresponds in molecular weight to SEMA5A. SEMA5A expression was low in non-metastatic 3T3 cells, yet increased in metastatic Ras-transformed 3T3 cells (FIG. 4A).

The ATX gene has been shown to amplify the invasive and metastatic potential of Ras-transformed cells (Nam, et al., Oncogene 19: 241-247, 2001). SEMA5A was further elevated in the Ras+ATX-transformed 3T3 cells. SEMA5A expression was studied in human tumor lines of defined metastatic phenotype (Inoue, et al., J. Cell Physiol. 156: 212-217. 1993). The 3T3, 3T3-RAS, and 3T3-RAS-ATX cells were previously characterized (Nam et al., 2000, supra). Mice injected subcutaneously with 3T3 cells developed, on average, 3 lung metastases (range 0-16) while mice injected with 3T3-RAS-ATX developed, on average, 80 lung metastases (range 10-200) and those injected with untransfected 3T3 cells did not develop lung metastases (Nam et al., 2000, supra). Highly metastatic MDA435 expressed greater levels of semaphorin compared to low metastatic potential MDA231 or non-metastatic A2058 cells (FIG. 4A). Using immunofluorescence, the Semaphorin protein was localized to the cell membrane (FIG. 4B) in MDA435 cells.

Sema-5c Mutation Disrupts Dpp Signaling in l(2)gl Homozygotes

Selected signal transduction pathway phosphoproteins were examined by reverse phase protein microarray analysis (linearity r=0.99, s.d.<10% of the mean). See, e.g., Paweletz, et al., Oncogene 20: 1981-1989, 2001 (FIG. 3C). Larval brain extracts were prepared as described for Western blotting. A serial dilution of each lysate was prepared. A total of 50 nl (5 nl applied in a series of 10 separate applications) of the lysate was arrayed with a “pin and ring” GMS 417 microarrayer (Affymetrix) using a 500-micron pin onto nitrocellulose slides with a glass backing (Schleicher and Schuell). Spatial densities of 980 spots/slide were achieved on a 20 mm×50 mm slide.

Staining was performed using a DAKO Immunostainer automated slide stainer using the Catalyzed Signal Amplification (CSA) system (Dako) as previously described (Paweletz et al 2001). Antibodies used were: anti-actin 1:250 (Oncogene), anti-P13K 1:100 (Cell Signaling), anti-T-ERK 1:500 (Cell Signaling), anti- P-ERK 1:1000 (Cell Signaling), anti-c-caspase 3 1:500 (Cell Signaling), anti-SMAD1 1:100 (Santa Cruz Biotechnology), and anti-P-SMAD1 1:250 (Cell Signaling). Cross reaction of the anti-human P-SMAD 1 to Drosophila phospho-Mad was verified by treatment of disaggregated fly cells with 40 μg/ml dpp protein (R&D Systems). Specificity of each antibody was validated by detecting a single band by Western blotting. Arrays were scanned with an Epson Perfection 1640SU scanner using Adobe PhotoShop 5.5 at a resolution of 1200 dpi and analyzed with ImageQuant (Molecular Dynamics).

The levels of Mothers against dpp (Mad), P13K, ERK, Akt, and cleaved caspase 3 were studied in brain extracts from l(2)gl/l(2)gl, l(2)gl/l(2)gl; sema-5c/sema-5c, and wild-type larvae. P13K was reduced in l(2)gl/l(2)gl;sema-5c/sema-5c compared to l(2)gl/l(2)gl. To further study the role of P13K in l(2)gl tumors, the P13K inhibitor, LY294002, was orally administered to Drosophila adults injected with l(2)gl/l(2)gl tissue. LY294002 treatment reduced the primary tumor size to 7% of untreated hosts, without adverse effects to the hosts (data not shown).

The largest difference observed by protein microarray analysis between l(2)gl/l(2)gl and l(2)gl/l(2)gl; sema-5c/sema-5c larval brain protein extracts was in levels of phospho-Mad. P-Mad was overexpressed in l(2)gl/l(2)gl compared to wild-type tissues. Following loss of sema-5c, P-Mad levels were reduced below the wild-type.

The sema-5c gene is shown here for the first time to be absolutely required for growth and metastasis of l(2)gl tumors. The absence of sema-5c in the mutant line completely blocked tumorigenesis and metastasis and reversion of the mutation recovered the malignant phenotype. The expression of the sema-5c homolog, SEMA5A, correlated with metastatic potential in 3T3, Ras-3T3, and Ras-ATX 3T3 cell lines. SEMA5A levels also correlated with metastatic potential in human breast carcinoma and melanoma cell lines (FIG. 4A). This suggests that class 5 semaphorins may also play a role in mammalian tumorigenesis and metastasis.

### Example 2

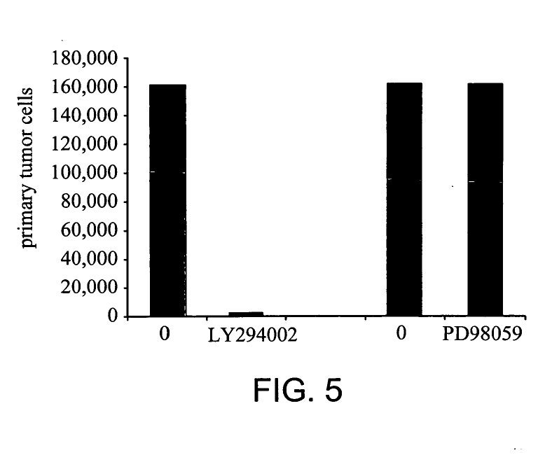

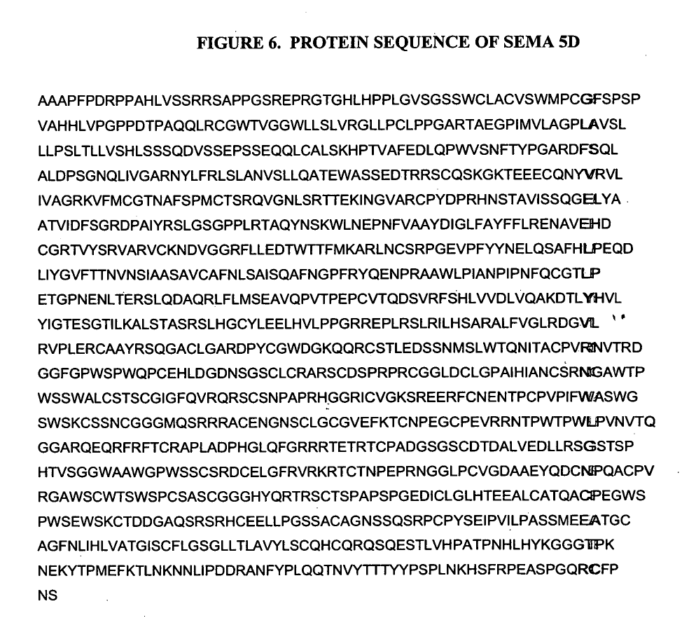

Adult βgal^{nl} hosts transplanted with armadillo-lacZ marked l(2)gl brain fragments were treated with 0; 0.556; 5.56; and 55.6 μg/ml of the PI-3 K inhibitor, LY294002 (Sigma), by adding drug to fly media. Flies were cultured for 21 days on drug-containing food and stained for the presence of β-galactosidase. Primary tumor size was determined by counting the cells dissociated from tumors. See, e.g., FIG. 5.

Variations, modifications, and other implementations of what is described herein will occur to those of ordinary skill in the art without departing from the spirit and scope of the invention and claims.

All of the patents, patent applications, international applications, and references described above are incorporated by reference herein in their entireties.

1. A method of identifying the risk of abnormal cellular proliferation in a subject, comprising: detecting expression of a Sema 5 gene product,
wherein overexpression of the Sema 5 gene product compared to expression of Sema 5 gene product in a reference sample from a subject with normally proliferating cells, provides an indication of increased risk of the presence of abnormal cellular proliferation in the subject:

2. The method according to claim 1, wherein the subject is a human being.

3. The method of claim 1, wherein the increased risk is associated with cancer.

4. The method of claim 1, wherein detecting is performed by obtaining a sample from the subject, contacting the sample with a molecular probe which specifically binds to a Sema 5 gene product, and identifying the presence and/or amount of binding complexes formed between the molecular probe and Sema 5 gene product.

5. The method of claim 1, wherein the Sema 5 gene product comprises a nucleic acid.

6. The method of claim 1, wherein the Sema 5 gene product comprises a polypeptide.

7. The method of claim 4, wherein the molecular probe comprises an antibody or antigen binding fragment thereof.

8. The method of claim 4, wherein the molecular probe comprises a nucleic acid.

9. A method of screening for a subject at risk for developing or having cancer resulting from abnormally proliferating cells comprising an altered or misexpressed Sema 5 gene product or an amplified Sema 5 gene, the method comprising the steps of:
obtaining cells from an individual at risk for or having cancer and detecting the presence of an altered or misexpressed Sema 5 gene product or amplified Sema 5 gene; and
correlating the presence of the altered or misexpressed Sema 5 gene product or amplified Sema 5 gene with risk for developing or having cancer.

10. A method of screening for polymorphisms in a Sema 5 gene associated with increased risk of having or developing cancer, comprising obtaining a biological sample from a subject with an increased risk for developing cancer or having cancer, and identifying the presence or absence of a polymorphism in a Sema 5 gene in the subject.

11. An isolated immune effector cell which specifically recognizes a SEMA 5 antigen.

12. A vaccine composition comprising at least one SEMA 5 antigen or nucleic acid molecule encoding at least one SEMA 5 antigen and an adjuvant for enhancing an immune response.

13. The vaccine composition according to claim 12, comprising a vaccine viral vector comprising the nucleic acid molecule.

14. The vaccine composition of claim 12, wherein the at least one SEMA 5 antigen comprises a plurality of different SEMA 5 antigens.

15. The method according to claim 12, wherein the adjuvant is a cytokine.

16. A therapeutic antibody composition comprising an antibody or antigen binding fragment thereof which specifically binds to a SEMA 5 antigen, wherein the antibody is stably associated with at least one effector molecule for targeting or killing a cell.

17. The antibody of claim 16, wherein the effector molecule comprises a toxin.

18. The antibody of claim 16, wherein the effector molecule comprises a molecule that specifically binds to a cancer cell.

19. A composition for treating abnormal cellular proliferation in a subject comprising administering a molecule that decreases or prevents expression of a SEMA 5 gene product.

20. The composition according to claim 19, wherein the molecule is an antisense molecule, a ribozyme, an iRNA, or an anti-SEMA 5 antibody.

21. A library of variant Sema 5 molecules.

22. A kit comprising a molecular probe which specifically binds to a Sema 5 gene product and a biological sample comprising an abnormally proliferating cell or a portion thereof which overexpresses SEMA 5.

23. The kit according to claim 22, wherein the biological sample comprises a cell lysate, a cell culture sample, or a tissue section.

24. The kit according claim 22, further comprising a biological sample comprising a normally proliferating cell or portion thereof.

25. A method of inhibiting cellular proliferation, comprising administering to a cell an effective amount of the composition of claim 11, 12, or 19 to inhibit cellular proliferation.

26. The method of claim 25, comprising administering the composition of claim 11, 12, or 19 to a subject in an amount effective to inhibit abnormal cellular proliferation.

27. The method of claim 26, wherein administering comprises topical administration or injection into a tumor.

28. The method of claim 25, wherein the subject is a human.

29. A method of generating an immune response against abnormally proliferating cells comprising administering to a subject, an effective amount of the composition of claim 11 or 12.

30. A method for identifying a mutated gene which is a modulator of a neoplastic phenotype, comprising:
introducing a tissue into an adult fly, wherein the tissue comprises a reporter sequence and a mutated modulator gene and wherein the tissue is derived from a mutant fly comprising a mutated l(2)gl gene capable of conferring a neoplastic phenotype and a mutated sema 5 gene suppressing the neoplastic phenotype of the mutated l(2)gl gene;
monitoring the expression of the reporter sequence in cells from different tissues in the adult fly, wherein one or more of: an increase in the numbers of different tissues expressing the reporter sequence and an increase in the level of reporter sequence expressed in one or more tissues, identifies the mutated modulator gene as a modulator of a neoplastic phenotype.

31. A method for identifying a mutated gene which is a modulator of a neoplastic phenotype, comprising:
introducing a tissue into an adult fly comprising at least one copy of a candidate mutated modulator gene, wherein the tissue comprises a reporter sequence and is derived from a mutant fly comprising a mutated l(2)gl gene capable of conferring a neoplastic phenotype and a mutated Sema 5 gene which suppresses the neoplastic phenotype of the mutated l(2)gl gene;
monitoring the expression of the reporter sequence in cells from different tissues in the adult fly, wherein one or more of: a change in the numbers of different tissues expressing the reporter sequence and an increase in the level of reporter sequence expressed in one or more tissues identifies the mutated modulator gene as a modulator of a neoplastic phenotype.

32. A method for identifying a mutated gene which is a modulator of a neoplastic phenotype, comprising:
introducing a tissue into an adult fly, wherein the tissue comprises a reporter sequence and overexpresses a Sema 5 gene;
monitoring the expression of the reporter sequence in cells from different tissues in the adult fly, wherein one or more of: a change in the numbers of different tissues expressing the reporter sequence and a change in the level of a reporter sequence expressed in one or more tissues, identifies the mutated modulator gene as a modulator of the neoplastic phenotype.

33. The method of claim 31, wherein the mutation in the l(2)gl gene is a null or hypomorphic mutation.

34. The method of claim 31, wherein the mutation in the Sema 5 gene is a functional null mutation, a hypomorphic mutation, neomorphic mutation, or a conditional mutaton.

35. The method of claim 31, wherein the tissue is obtained from one or more larvae.

36. The method of claim 35, wherein the tissue is brain tissue or imaginal disc tissue.

37. The method of claim 31, wherein the reporter sequence is comprised within a P-element.

38. The method of claim 31, wherein the reporter sequence is selected from the group consisting of : lacZ gene, GFP gene, BFP gene, and luciferase gene.

39. The method of claim 31, wherein the mutant fly is homozygous for the mutated l(2)gl gene.

40. The method of any of claims 30, wherein the mutant fly is homozygous for the mutated Sema 5 gene.
